(19) United States
(12) Patent Application Publication
(10) Pub. No.: US 2021/0040494 A1
(43) Pub. Date: Feb. 11, 2021

(54) NON-TRANSGENIC PLANTS WITH MUTATED GLUTAMATE DECARBOXLASES FOR AGRONOMIC BENEFITS

(71) Applicant: PLANT SENSORY SYSTEMS LLC, Baltimore, MD (US)

(72) Inventors: Frank J. TURANO, Baltimore, MD (US); Kathleen TURANO, Baltimore, MD (US)

(73) Assignee: PLANT SENSORY SYSTEMS LLC, Baltimore, MD (US)

(21) Appl. No.: 17/045,059

(22) PCT Filed: Apr. 6, 2018
(86) PCT No.: PCT/US2018/026531

§ 371 Date: Oct. 2, 2020

(51) Int. Cl. C12N 15/82

(52) U.S. Cl. CPC C12N 15/8242; C12N 15/8213

(57) ABSTRACT

The present invention describes a non-transgenic approach to produce truncated glutamate decarboxylase (GAD) genes and the corresponding truncated gene product in plants. More particularly, the invention relates to the removal of the calcium-calmodulin binding domain (CaM-BD) from plant GADs using site-directed mutagenesis or gene-editing techniques. The removal of the CaM-BD from plant GADS results in an active GAD enzyme that is not regulated by the CaM-BD, which increases gamma aminobutyric acid (GABA) production in plant cells, organs or whole plants. Non-transgenic plants with truncated GAD have agronomic benefits, including increased GABA, biomass, yield, sugar, and tolerance to abiotic and biotic stressors. In addition, GABA from plants could be used as nutraceutical, pharmaceutical, or therapeutic compounds or as a supplement in animal feed or for animal feed or as an enhancer for plant growth or yield.

### SEQUENCE SUBMISSION

The present application is being filed along with a Sequence Listing in electronic format. The Sequence Listing is entitled Mutated_GAD_Gene_Sequences2.txt, created on Mar. 29, 2018 and is 66 kb in size. The information in the electronic format of the Sequence Listing is incorporated herein by reference in their entirety.

### FIELD OF THE INVENTION

The present invention relates to the production of a non-transgenic plant with improved or enhanced agronomic traits that include increased sugar, yield or tolerance to abiotic or biotic stress by site-directed mutation(s) of one of more of the root-expressed glutamate decarboxylase (GAD) genes in a plant. The present invention also relates to increased accumulation of the non-amino acid gamma aminobutyric acid (GABA) in tissues or organs of the non-transgenic plant. The present invention also relates to the use of methods using oligonucleotides, recombinagenic oligonucleobases protein-nucleotide complexes or specialized nucleases to make a desired mutation in the plant nucleotide sequences of a plant gene encoding GAD to produce a truncated GAD gene product. The present invention also relates use of the said plant organs or extracts from the organs for use as feed, food, health supplement, pharmaceutical or therapeutic.

### BACKGROUND OF THE INVENTION

GABA in Plants

GABA is required for normal plant growth and development (1, 2) and reproduction (2). GABA rapidly increases in response to a variety of stresses (3-10) and plays a role in stress tolerance (1). Elevated levels of GABA in plants have been shown to provide agronomic benefits. Table 1 lists several studies that have reported positive effects of exogenous GABA on plants, including increased growth and development (11-13), tolerance to abiotic stresses (14-25), accumulation of organic and amino acids and sugars (23), and improved photosynthesis (13, 26) and nitrogen metabolism (13). Elevated endogenous GABA levels have been also shown to increase resistance to nematodes (27).

TABLE 1
Agronomic benefits of exogenous GABA to plants

| Benefit | Plant/crop | Reference |
|---|---|---|
| growth and development | longstalk starwort (Stellaria longipes) | Kathiresan et al., 1998 (11) |
| | duckweed (Lemna minor L.) | Kinnersley et al., 2000 (12) |
| | maize (Zea mays L.) | Li et al., 2016 (13) |
| oxidative stress tolerance | Barley (Hordeum vulgare L.) | Song et al., 2010 (14) |
| | maize (Zea mays L.) | Wang et al., 2017 (15) |
| chilling tolerance | peach fruits (mygdalus persica) | Schirra et al., 2011 (16) |
| | anthurium cut flowers | Yang et al., 2011 (17) |
| | tomato seedling (Lycopersicon esculentum) | Aghdam et al., 2016 (18) |
| | wheat (Triticum aestivum) | Malekzadeh et al., 2014 (19) |
| salt tolerance | maize (Zea mays L.) | Wang et al., 2017 (15) |
| drought tolerance | perennial ryegrass (Lolium perenne) | Krishnan et al., 2013 (20) |
| | white clover (Trifolium repens) | Yong et al., 2017 (21) |
| osmotic tolerance | black pepper (Piper nigrum Linn.) | Vijayakumari et al., 2016 (22) |
| heat tolerance | creeping bentgrass (Agrostis stolonifera) | Li et al., 2016 (23) |
| | rice (Oryza sativa L.) | Nayyar et al., 2014 (24) |
| heavy metal tolerance | mustard (Brassica juncea L.) | Mahmud et al., 2017 (25) |
| accumulation of organic acids | creeping bentgrass (Agrostis stolonifera) | Li et al., 2016 (23) |
| accumulation of amino acids | creeping bentgrass (Agrostis stolonifera) | Li et al., 2016 (23) |
| accumulation of sugars | creeping bentgrass (Agrostis stolonifera) | Li et al., 2016 (23) |
| improved photosynthesis | maize (Zea mays L.) | Li et al., 2016 (13) |
| | wheat | Li et al., 2016 (26) |
| nitrogen metabolism | maize | Li et al., 2016 (13) |

GABA levels in plants are regulated endogenously by one of three metabolic pathways: the catabolism of polyamines (28, 29), the GABA shunt (2, 30, 31), and the decarboxylation of glutamate by the GAD enzyme. GAD is the major GABA-producing pathway (32).

GAD is a small gene family that contains three to ten members in most plants, with each member having a distinct gene expression profile. In plants, nearly all GAD activity is controlled (33) by the binding of calcium (Ca²⁺) and calmodulin (CaM) to a 22 to 40 amino acid region (34) at the carboxy-terminus of the protein (27), called a calmodulin-binding domain (CaM-BD) (34). The CaM-BD functions as an autoinhibitory domain to inactivate the GAD enzyme (35). The CaM-binding domain has three positively charged regions. The second region, a lysine-rich region, contains a conserved tryptophan (W). In some plants, these regions are missing or, if present, are significantly divergent in their amino acid composition. For example, one or more GADs in Arabidopsis, rice, soybean, maize, apple and tea do not contain complete positively-charged regions. The rice GAD2, which is lacking in some of these regions, does not bind Ca²⁺/CaM (36). Apple GAD3 is not regulated by Ca²⁺/CaM and is not inhibited by its C-terminus.

GAD enzymes that contain a CaM-BD can be deregulated by removing the CaM-BD to create an active truncated GAD (truncGAD) (35, 37-39), which results in increased GABA production in plant cells (27, 35, 37) and low glutamate (Glu) levels (35, 40, 41). Overexpression of truncGAD in plants has been shown to provide increased protection against biotic stresses, including resistance to the northern root nematode (27).

Plants with constitutively expressed truncGADs with very high levels of GABA have been associated with morphological abnormalities (35, 37), dwarfism and sterility (37). One study reported no morphological deformities (27) in plants that had a constitutively expressed truncGAD with moderate increases in GABA.

It is known that plants have tissue-specific or tissue-preferred GAD expression. For example, root-specific or root-preferred GAD expression has been demonstrated in Arabidopsis (42, 43) and fruit-specific or fruit-preferred GAD expression has been demonstrated in tomato (39).

Targeted expression of truncGAD in specific tissues has produced diverse results with respect to GABA levels and negative side effects (38, 39). For example, the expression of truncGAD in rice using an endosperm-specific promoter resulted in seeds with high GABA levels and no reported abnormalities (38). In another study, two different tomato fruit-expressed GAD genes (GAD2 and GAD3) were truncated using site-specific mutagenesis (39). TruncGAD2 plants with high GABA levels in the leaves had suppressed plant growth, flowering, and fruit yield, whereas truncGAD3 plants had elevated GABA levels in fruits but not in leaves, and plant size was less affected than that of the truncGAD2 plants (39). The authors hypothesized that the phenotypic differences could be due to differences in the gene expression profiles of the GAD2 and GAD3 genes, wherein the latter has higher expression in fruit (39). Expression of a truncGAD3 in tomato using a fruit-specific promoter resulted in normal plants but the plants did not form red-ripe fruits (44).

Plants with elevated levels of endogenous GABA, which was increased by means other than the GAD pathway, namely by the insertion of a bacterial polyamine pathway (45-47) or by the promiscuous activity of SAD (48), have increased GABA, yield, biomass and abiotic stress tolerance (45-47).

In order to achieve the positive agronomic effects of increased GABA without the negative side effects, the present invention produces a truncGAD, preferably the production of truncGAD in roots using a gene editing technology. For purposes of the present invention members of the GAD family were identified and root-specific or root-preferred GADs were selected in sugar beet, soybean, rice, and corn. The present invention mutates a root-preferred GAD protein in sugar beet to produce a truncGAD plant that has increased sugar content and root biomass. The present mutated truncGAD was changed at the carboxyl end of the GAD protein to insert a stop codon and remove portions or the entire CaM-BD in beet. The present invention relates to the production of truncGAD in any plant by mutation at analogous amino acid residues in a GAD homolog, ortholog or paralog that contains a CaM-BD.

The approaches described herein can be used by those of ordinary skill in the art to identify and mutate any plant GAD gene to make a truncGAD. In addition, plant GADs with specific or preferred expression in other plant tissues, including stems, stalks, stolons, leaves, flower, petioles, cotyledons, petioles, buds, stamens, pods, grain, fruit, nuts, seed or husks, can be mutated to produce a truncGAD. The approaches described herein can be used by those of ordinary skill in the art to identify and mutate GAD genes that are expressed in specific tissues other than root. In addition, the approaches described herein can be used by those of ordinary skill in the art to identity and remove the carboxyl terminus of a plant GAD that extends past the GAD enzymatic region, such as those identified in rice GAD2 and apple GAD3.

Site-Specific Gene Editing Technologies

Site-specific mutagenesis or genome editing protocols are utilized in the present invention and are known to those with ordinary skill in the art. Genome editing technologies have been successfully used in plants and include zinc finger nucleases (ZFNs) (49-52), Transcription Activator-Like Effector Nucleases (TALENs) (53), Clustered Regularly Interspaced Short Palindromic Repeats and associated proteins (CRISPR/Cas) (54-56), and oligonucleotide-directed mutagenesis (ODM) (57-59). For an overview of genome editing technologies used in plants see (60-63) and references therein. The protocols are similar in function in that they recognize specific nucleotide targets and replace the target DNA sequence with a specific nucleotide or a polynucleotide sequence (64). The protocols differ in their components and/or structure. Some systems are composed of proteins or protein complexes, such as ZFNs and TALENs. Other systems are composed of nucleotides (dideoxynucleotide or ribonucleotide)/protein complexes, such as the CRISPR system. Other systems, such as ODM, use only nucleotides, ribonucleotides or modified bases.

Recently, higher genome-editing frequency was obtained by the combination of the TALENS or CRISPR/Cas9 systems with the ODM technology. The TALENS or CRISPR/Cas9 systems utilize site-specific DNA double-strand breaking nucleases, and the ODM technology produces random breaks using the DNA double strand breaking systems like members of the bleomycin family of glycopeptide antibiotics that include but are not limited to the antibiotic, phleomycin (65).

ZFNs, which are composed of an array of zinc finger domains, are capable of recognizing a target sequence of six to nine consecutive nucleotides. Fused on the C-terminus end of the recognition DNA is a nuclease domain of the FokI endonuclease. A second array of zinc finger domains, capable of recognizing a target sequence on the opposite strand and also fused with a FokI, places the two FokI nucleases in close proximity to form a functional dimer that cleaves the target DNA molecule. (66, 67). The break is repaired by the nonhomologous end joining (NHEJ) DNA repair mechanism, which inserts or deletes nucleotides. Alternatively, the break in the DNA fragments can be repaired by homologous recombination (HR) using DNA fragments with homologous regions to the cleavage site to make site-specific nucleotide insertions or deletions (68).

TALENs are similar to ZFNs in that they are composed of a fusion of a DNA recognition domain and a nuclease domain. The main difference between the two technologies is that the DNA recognition domain in TALENs is made up of repeats derived from transcription activator-like (TAL) effectors (64, 69). The repeats are highly conserved except for two highly variable amino acid residues that are involved in binding to specific nucleotide bases. Manipulation of the repeats with distinct residues at the variable amino acid residues allows for the assembly of a DNA sequence to quickly and efficiently alter genes (53, 70). Similar to the ZFNs system, the break in the DNA can be repaired by the NHEJ or the HR system if a DNA segment with homologous ends to the cleavage site are present. TALENs has been successfully used in Oryza sativa (71).

The CRISPR/Cas system is a site-specific gene editing tool known for its easy manipulation and high efficiency (72). Of the three types of CRISPR/Cas (I, II and III) (73), type II CRISPR/Cas9 is the one initially demonstrated to work in plants (54-56) and the one most widely used today. CRISPR/Cas9 vectors or plasmids for use in plants are available from various non-profit organizations and manufacturers. The CRISPR system utilizes a guide or guiding RNA (gRNA or sgRNA) molecule that is homologous to the sequence of the target site with the exception of the desired mutation(s) for recognition of the sequence. Cas9 is involved in DNA cleavage at the target site, which promotes the endogenous DNA repair system to incorporate the gene mutation. The RNA molecule can be modified to recognize specific genome target sequences. Similar to the ZFNs and TALENs systems, the break in the DNA can be repaired by the NHEJ or by the HR system if a DNA segment with homologous ends to the cleavage site are present.

ODMs (57, 74) utilize synthetic single-stranded oligonucleotides (ssODNs) that are 41 to 201 nucleotides in length (also called chimeraplasts or recombinagenic oligonucleobases). The recognition portion of the molecule is composed of 20 to 40 DNA and RNA nucleotides that are homologous to the sequence of the target site with the exception of the desired mutation(s) for recognition of the sequence. The recognition portion of the molecule forms a duplex or hairpin structure through complementary nucleotides in the 3′ and 5′ regions of the molecule. The mismatched (mutation) recognition portion of the ssODNs is incorporated into the genome by the endogenous DNA repair system. Other oligonucleotide modifications include 2′-O-methyl-modified RNA bases for higher binding affinity to the target and modifications of the terminal bases with phosphorothioate linkages or modification of the 5′ end with Cy3. The fluorescent dye, tetramethylrhodamine, and the 3′ end with reverse base or inverted base C (idC), have been used to stabilize the molecule. Other dyes can also be used, such as the indocarbocyanine dye as described in U.S. Pat. Nos. 5,556,959 and 5,808,044. The ssODNs can also be protected from RNases by replacing the 2′-hydroxyl with other molecules that include, but are not limited to, the addition of bromo, chloro or fluor and other 2′-substituted nucleotides as described by Gocal et al. (57) and references therein.

Homing endonucleases (HEs) are very large sequence-specific endonucleases that are similar to a restriction enzyme but recognize and cut longer target sequences (>14 bp) (75, 76). HEs can be engineered to target specific DNA sites, and coupled with the endonuclease activity, may be a new tool that can be used to practice this invention.

Specific details for the design and use of the genome and gene editing tools are well known to those of ordinary skill in the art and can be used to mutate a plant GAD gene according to the present invention to make a truncGAD. The present invention also relates to mutations in any plant GAD to remove the C-terminal region to control GAD activity in the plant to obtain beneficial agronomic traits.

### SUMMARY

The present invention describes methods to produce truncated GAD peptides using a site-specific mutation of the plant GAD gene. The invention describes the specific targets for mutations of the GAD genes that are specifically, preferably or predominantly expressed in the roots. The invention describes the use of nucleotide, ribonucleotides, or nucleases to produce truncated GADs. The methods of site-directed mutagenesis in plants are well known to those in the art. The present invention is unique in that it describes how to produce a plant that, by specifically targeting GADs expressed in the root, exhibits one or more beneficial traits associated with elevated GABA levels in plants or plant cells, including increased tolerance to heat, salt, oxidative stress, insect feeding, nematode infection, photosynthetic capacity, sugars, biomass, or yield, and yet does not exhibit the negative side effects associated with high GABA levels.

In one embodiment a stop codon is introduced into at least one GAD DNA or nucleotide sequence in a plant cell or plant to remove the CaM-BD. In some embodiments, a stop codon is introduced into two or more of the DNA or nucleotide sequences of different GAD genes in a plant cell or plant to remove the CaM-BD.

### BRIEF DESCRIPTION OF THE FIGURES

FIG. 1. Alignment of GADs from E. coli, petunia and tobacco (truncated and non-truncated) with root-preferred GADs from sugar beet, soybean, rice, and maize to identify the positions of the CaM-BDs. The three positively charged regions (+R1, +R2 and +R3) of the CaM-BD are indicated by double underlines. K- and R-rich regions are indicated by vertical lines (1). The conserved W involved in CaM/Ca2+ binding is indicated in bold typeface. Dashes represent spaces to maximize amino acid alignment or missing amino acid residues. FL=full-length GAD.

FIG. 2. GAD gene products that are preferentially expressed in roots of Beta vulgaris (sugar beet). The underlined amino acid residues located in the carboxyl-region of the GAD peptides indicate the region where a mutation (stop codon) is incorporated into the corresponding nucleotide sequence of the gene to remove the CaM-BD and produce a truncGAD. The preferred location for the stop mutation is indicated with a box. A) XP_010690484; B) XP_010687254

FIG. 3. GAD gene product that is preferentially expressed in roots of Glycine max (soybean). The underlined amino acid residues indicate locations in the carboxyl-region of the GAD peptide where a mutation (stop codon) can be incorporated into the corresponding nucleotide sequence of the gene to remove the CaM-BD and make a truncGAD. The preferred location for the stop mutation is indicated with a box.

FIG. 4. GAD gene product that is preferentially expressed in roots of Oryza sativa (rice). The underlined amino acid residues indicate locations in the carboxyl-region of the GAD peptide where a mutation (stop codon) can be incorporated into the corresponding nucleotide sequence of the gene to remove the CaM-BD and make a truncGAD. The preferred location for the stop mutation is indicated with a box.

FIG. 5. GAD gene products that are preferentially expressed in roots of Zea mays (maize or corn). The underlined amino acid residues indicate locations in the carboxyl-region of the GAD peptide where a mutation (stop codon) can be incorporated into the corresponding nucleotide sequences of the genes to remove the CaM-BD and make a truncGAD. The preferred location for the stop mutation is indicated with a box. A): NP_001167941; B) NP_001130451.

FIG. 6. Identification of non-transgenic sugar beet plants that contain a truncGAD. PCR analysis was performed on DNA isolated from the leaves of 30-day-old sugar beets, using mutant-specific primers. The resulting amplified DNA fragments were separated and visualized in a 1.5% agarose gel.

### DETAILED DESCRIPTION OF THE INVENTION

To date, all plants whose genomes have been sequenced and reported have more than one GAD gene. Most of the plant GADs contain a carboxyl CaM-BD that negatively regulates GAD activity. In the present invention one or more of the GAD genes in a plant can be mutated to make a truncGAD. In one embodiment of the invention all of the GAD genes in a plant can be mutated to make truncGADs. In another embodiment GAD genes with CaM-BD are mutated to remove the CaM-BD domain to produce a truncGAD. In a preferred embodiment, GAD genes with CaM-BD that are specifically or preferentially expressed in roots are mutated to remove the CaM-BD domain to produce a truncGAD. In practicing the present invention site-specific mutagenesis or genome editing protocols known to those of ordinary skill in the art are utilized to replace an amino acid codon with a stop codon or to insert a stop codon to produce a truncGAD.

Identification of Plant GADs

In practicing the present invention, GAD sequences can be identified in gene databases. The databases include but are not limited to NCBI, KEGG, EMBL or plant species-specific databases. The BLAST family of programs can be used for nucleotide or protein database similarity searches. BLASTN searches a nucleotide database using a nucleotide query. BLASTP searches a protein database using a protein query. BLASTX searches a protein database using a translated nucleotide query that is derived from a six-frame translation of the nucleotide query sequence (both strands). TBLASTN searches a translated nucleotide database using a protein query that is derived by reverse-translation. TBLASTX searches a translated nucleotide database using a translated nucleotide query.

BLASTP searches can be performed using a protein database using a protein query. Examples of GAD amino acid sequences that can be used for BLASTP in the sequence listing: SEQ ID NO:1, SEQ ID NO:2, SEQ ID NO:3, SEQ ID NO:4, SEQ ID NO:5, SEQ ID NO:6 SEQ ID NO:7, SEQ ID NO:8, SEQ ID NO:9 and SEQ ID NO:10. The invention is not limited to the use of these amino acid sequences. GAD amino acid sequences from other species with substantial identity can be used. Those of ordinary skill in the art know that organisms of a wide variety of species commonly express and utilize homologous proteins, which include the insertions, substitutions and/or deletions discussed above, and effectively provide similar function. For example, the amino acid sequences for GAD from Petunia hybrida, Nicotiana tabacum, Beta vulgaris, Glycine max, Oryza sativa, or Zea mays may differ to a certain degree from the amino acid sequences of GAD in another species and yet have similar functionality with respect to catalytic and regulatory function. Amino acid sequences comprising such variations are included within the scope of the present invention and are considered substantially or sufficiently similar to a reference amino acid sequence. Although it is not intended that the present invention be limited by any theory by which it achieves its advantageous result, it is believed that the identity between amino acid sequences that is necessary to maintain proper functionality is related to maintenance of the tertiary structure of the polypeptide such that specific interactive sequences will be properly located and will have the desired activity, and it is contemplated that a polypeptide including these interactive sequences in proper spatial context will have activity.

Unless otherwise stated, sequence identity or similarity values refer to the value obtained using the BLAST 2.0 suite of programs using default parameters (77). As those of ordinary skill in the art understand that BLAST searches assume that proteins can be modeled as random sequences and that proteins comprise regions of nonrandom sequences, short repeats, or enriched for one or more amino acid residues, called low-complexity regions. These low-complexity regions may be aligned between unrelated proteins even though other regions of the protein are entirely dissimilar. Those of ordinary skill in the art can use low-complexity filter programs to reduce number of low-complexity regions that are aligned in a search. These filter programs include, but are not limited to, the SEG (78, 79) and XNU (80).

The invention is not limited to the identification of GADs by database searches. Those of ordinary skill in the art know that GAD genes or the corresponding RNA sequences can be identified by molecular or biochemical methods. The methods include but are not limited to screening of DNA or cDNA libraries using DNA hybridization, immuno (western) blot analysis, or PCR-based screening with degenerated primers to conserved GAD domains. Once the putative GAD genes are identified, they can be confirmed by DNA sequencing. Suitable nucleotides for probes for the hybridization or PCR-based screens include but are not limited to the corresponding nucleotide sequences for GAD peptides from Petunia hybrida (SEQ ID NO:1), Nicotiana tabacum (SEQ ID NO:2), sugar beet (SEQ ID NO:3 [XP_010690484]; SEQ ID NO:4 [XP010687254]), soybean (SEQ ID NO:6 [NP_001276214]), rice (SEQ ID NO:7 [XP_015631723]) and maize (SEQ ID NO:9 [NP_001130451]; SEQ ID NO:10 [NP_001167941]). Those of ordinary skill in the art know that the corresponding GAD nucleotide sequence for any peptide is available on publicly available databases, such as NCBI. Table 2 lists the corresponding cDNA GenBank Accession numbers for SEQ ID NOs. 1 through 10. It is known to those of ordinary skill in the art that hybridization conditions can be modified so that nucleotides from a known GAD gene or cDNA can selectively hybridize to heterologous genes. Those of ordinary skill in the art know that GAD genes can also be identified by DNA-seq, RNA-seq or exome sequence analyses.

TABLE 2
Corresponding cDNA for SEQ ID Nos. 1-10

| | | GenBank |
|---|---|---|
| | SEQ ID NO | ACCESSION |
| Genus Species (common name) | Peptide | Encoding cDNA |
| Petunia hybrida (petunia) | SEQ ID NO: 1 | L16977.1 |
| Nicotiana tabacum (tobacco) | SEQ ID NO: 2 | NM_001325840 |
| Beta vulgaris (sugar beet) | SEQ ID NO: 3 | XM_010692182 |
| Beta vulgaris (sugar beet) | SEQ ID NO: 4 | XM_010688952 |
| Glycine max (soybean) | SEQ ID NO: 5 | XM_003531075 |
| Glycine max (soybean) | SEQ ID NO: 6 | NM_001289285 |
| Oryza sativa (rice) | SEQ ID NO: 7 | XM_015776237 |
| Oryza sativa (rice) | SEQ ID NO: 8 | XM_015778429 |
| Zea mays (maize) | SEQ ID NO: 9 | NM_001136979 |
| Zea mays (maize) | SEQ ID NO: 10 | NM_001174470 |

Identification of Tissue Specific GAD

Gene expression profile databases such as GEO, Genevestigator, Tissue-specific Gene Expression and Regulation (TiGER), PLEXdb (Plant Expression Database), and Expression Atlas can be used to determine the gene expression profiles of plant genes. Protein sequences can be used to search NCBI for homologs, orthologs or paralogs. In addition, mRNA sequences for the peptides can be obtained through the link “Encoding mRNA”. Gene expression profiles can be obtained through the link to “Gene”, followed by the link to “GEO Profiles”. In addition, there are species-specific genome databases and many of these databases contain gene expression databases that can be searched to identify gene expression profiles for a specific gene. These genome databases include but are not limited to SoyXpress, SoyBase, RiceXPro, and MaizeGDB. The invention is not limited to the use of these expression profile databases for the determination of the gene expression pattern of a specific gene. Those of ordinary skill in the art know that gene expression patterns, including temporal or spatial (tissue-specific) expression or expression due to environmental or biotic cues can be determined by molecular or biochemical methods. The methods include but are not limited to analyses of samples from different plant tissues using northern (RNA) blot analysis, immuno (western) blot analysis, 2-D gel electrophoresis, enzyme-linked immunosorbent assay (ELISA), Reverse Transcription PCR (RT-PCR), quantitative RT-PCR (qRT-PCR), expressed sequence tags (EST), RNA-Seq (RNA sequencing), whole transcriptome shotgun sequencing (WTSS), microarray analyses or with proteomic analyses using MALDI-TOF-MS-based mass pattern and fingerprinting, LC-MS/MS-based peptide sequencing.

Identification of the Region of the GAD to be Truncated

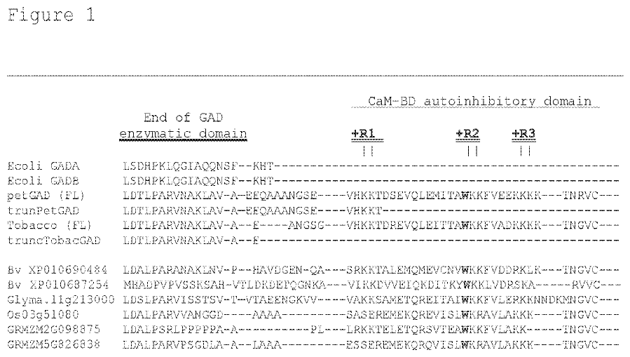

In practicing the present invention sequence alignment or multiple sequence alignments can be used to identify the CaM-BD of a GAD. Methods for sequence alignment are well known to those with ordinary skill in the art. The identified sequences for the genes or gene products can be aligned to identify the CaM-BD in a plant GAD and determine the location for the insertion of the stop codon to make a truncGAD. Methods for the alignment of nucleotide and amino acid sequences for comparison are well known to those of ordinary skill in the art. CLUSTAL Omega, DbClustal, KALIGN, COBALT, MAFFT and MULTALIN allow for the alignment of multiple sequences. Any of the above-mentioned alignment programs can be used to align protein sequences. As demonstrated in FIG. 1 plant GADs with known CaM-BDs were aligned with root-specific or -preferred CaM-BD-containing GADs from sugar beet, soybean, rice and corn, and with GADs without a CaM-BD, namely, E. coli GADA and GADB and the truncGADs from petunia and tobacco. The GADs in FIG. 1 include full-length GADs from Petunia hybrida (SEQ ID NO:1), Nicotiana tabacum (SEQ ID NO:2), root-specific or -preferred CaM-BD-containing GADs from sugar beet (SEQ ID NO:3 [XP_010690484]; SEQ ID NO:4 [XP_010687254]), soybean (SEQ ID NO:6 [NP_001276214]), rice (SEQ ID NO:7 [XP_015631723]) and maize (SEQ ID NO:9 [NP_001130451]; SEQ ID NO:10 [NP_001167941]), and GADs without a CaM-BD from E. coli (GADA SEQ ID NO:11; GADB SEQ ID NO:12), and truncGAD from petunia (SEQ ID NO:13) and tobacco (SEQ ID NO:14).

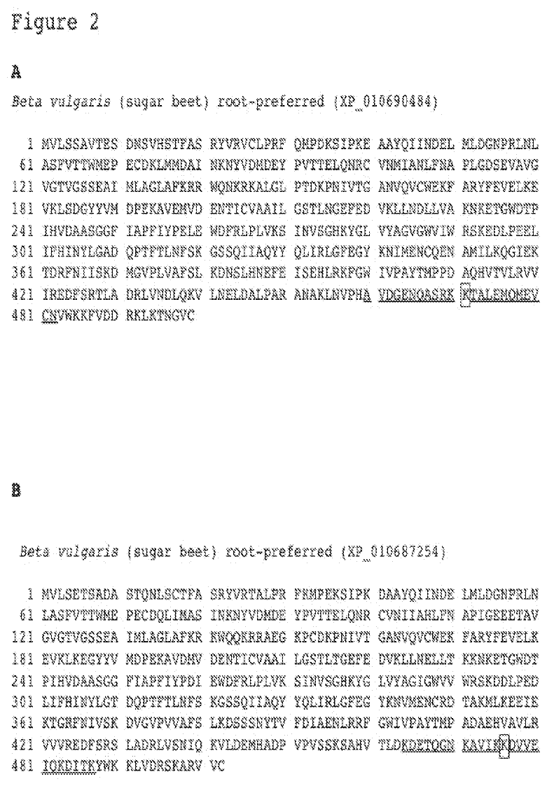

The length or size of the CaM-BDs in plant GAD genes varies both within and between species (FIGS. 1-5 and Table 3). These differences must be accounted for when making mutants according to the present invention. To make the mutated truncGAD, the analogous location of the amino acids in the CaM-BD of the GAD first needs to be identified. For example, the mutation that converts K471 into a stop in sugar beet GAD XP_010690484 is equivalent to the mutation that converts K476 mutation into a stop in sugar beet GAD XP_010687254. In addition, the position of the stop mutations can be located more towards the amino terminus or carboxyl terminus of the preferred location (boxed residues in FIGS. 2-5) by at least 7 to 10 residues, preferably 2 to 6 residues, and most preferably by one residue or at the site indicated (boxed residue) to produce a truncGAD. In the present invention a truncGAD is produced by the removal of the last 17 to 44 amino acid residues of a plant GAD. In the preferred embodiment the last 17 to 44 amino acid residues encode for the CaM-BD.

Sugar beet has five GAD genes (XP_010690484, XP_010687254, XP_010680063, XP_010680060 and XP_010680059). The amino acid sequences for two root-preferred GAD genes and the preferred locations of the stop codons for the formation of a truncGAD are identified in FIG. 2.

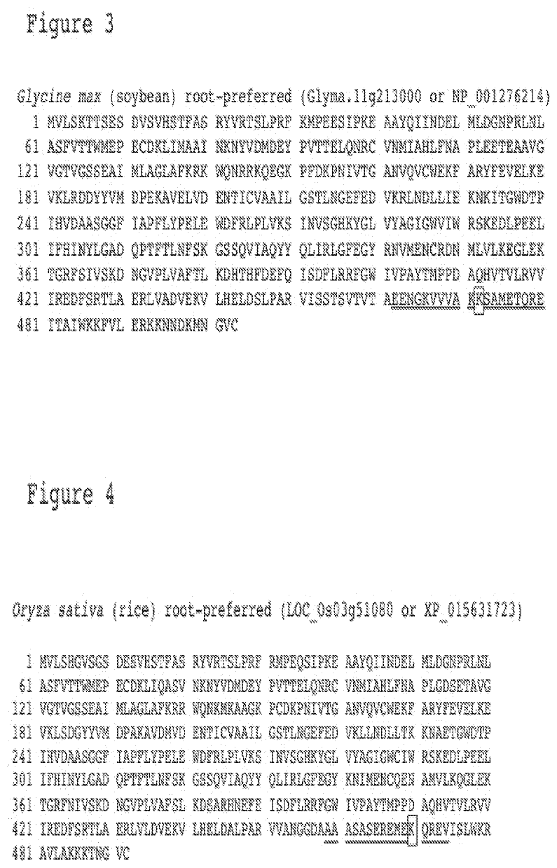

Soybean has ten GAD genes (Glyma.14G211100, Glyma.02G241400, Glyma.18G043600, Glyma.09G168900, Glyma.16G218500, Glyma.08G091500, Glyma.05G136100, Glyma.08G091400, Glyma.11G213000 and Glyma.08G091300). The amino acid sequences for one root-preferred GAD gene and the preferred location of the stop codons for the formation of a truncGAD are identified in FIG. 3.

Rice has five GAD genes (LOC_Os03g51080, LOC_Os03g13300, LOC_Os08g36320, LOC_Os04g37460 and LOC_Os04g37500). The amino acid sequences for one root-preferred GAD gene and the preferred locations of the stop codons for the formation of a truncGAD are identified in FIG. 4.

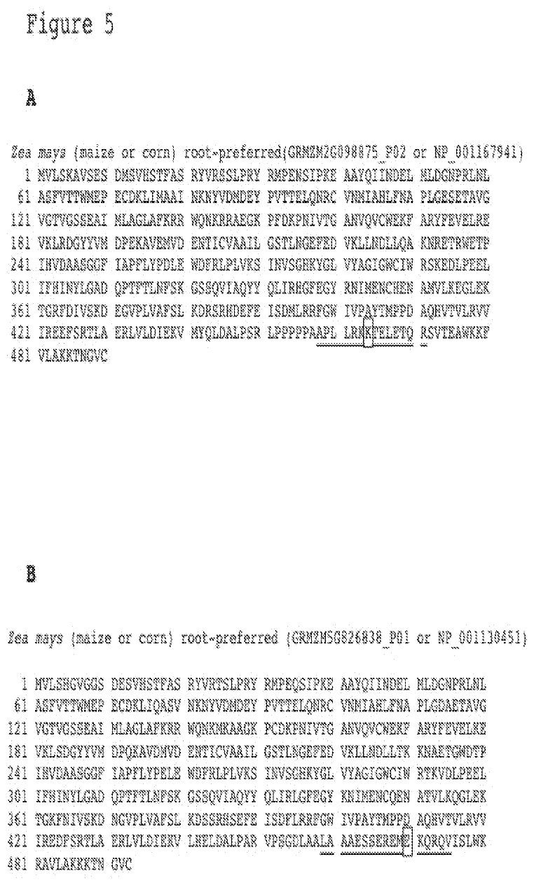

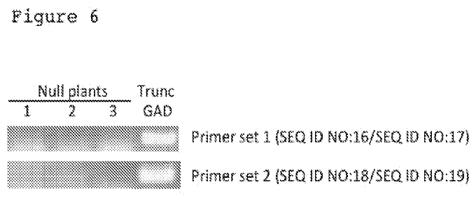

Maize has five GAD genes (GRMZM5G826838, GRMZM2G098875, GRMZM2G017110, GRMZM2G101069, and GRMZM2G355906). The amino acid sequences for two root-preferred GAD genes and the preferred locations of the stop codons for the formation of a truncGAD are identified in FIG. 5.

In the present invention codons for amino acid positions in a GAD gene from different plant species are mutated into stop codons to remove 17 to 44 amino acids of the CaM-BD to produce a truncGAD. Table 3 includes the positions of the amino acid, by amino acid number, for GAD from various species that can be the substituted with a stop codon to make a truncGAD. Mutations that convert the corresponding codon of any of the amino acid residues into a stop codon indicated in Table 3 or amino acid substitutions in any of the identified amino acid positions produce a truncGAD.

Table 3 lists a range of amino acid locations where a single amino acid codon can be mutated into a stop codon in the root-specific or root-preferred GAD genes (Gene ID or Genbank number) for preferred crops. In each case the codon for the amino acid (standard amino acid designations) substitution is listed to the right of the amino acid position number.

TABLE 3
Amino acid positions that can be converted to
a stop codon to make a functional truncGAD

| | | Amino acid positions |
|---|---|---|
| | | that can be converted |
| | | to a stop codon |
| | | to make a |
| Plant (Species) | Gene ID | functional truncGAD |
| Sugar beet (Beta vulgaris) | XP_010690484 | A460 to N482 |
| Sugar beet (Beta vulgaris) | XP_010687254 | K464 to K487 |
| Soybean (Glycine max) | NP_001276214 | E462 to E480 |
| Rice (Oryza sativa) | XP_015631723 | A459 to V474 |
| Maize (Zea mays) | NP_001167941 | A458 to R471 |
| Maize (Zea mays) | NP_001130451 | L459 to V475 |

Any of the following amino acids in the root-specific or root-preferred GADs in sugar beet can be changed to stop codons to practice the invention.
Sugar beet (Beta vulgaris) from the DNA translation of XP_01069048 into amino acids 1 to 499:
A460, V461, D462, G463, E464, N465, Q466, A467, 5468, R469, K470, K471, T472, A473, L474, E475, M476, Q477, M478, E479, V480, C481, or N482.
Sugar beet (Beta vulgaris) from the DNA translation of XP_010687254 into amino acids 1 to 502:
K464, D465, E466, T467, Q468, G469, N470, K471, A472, V473, 1474, K475, K476, D477, V478, V479, E480, 1481, Q482, K483, D484, 1485, T486, or K487.

Any of the following amino acids in the root-specific or root-preferred GAD in soybean can be changed to stop codons to practice the invention.
Soybean (Glycine max) from the DNA translation of NP_001276214 into amino acids 1 to 503:
E462, E463, N464, G465, K466, V467, V468, V469, A470, K471, K472, 5473, A474, M475, E476, T477, Q478, R479, or E480.

Any of the following amino acids in the root-specific or root-preferred GAD in rice can be changed to stop codons to practice the invention.
Rice (Oryza sativa) from the DNA translation of XP_015631723 into amino acids 1 to 492:
A459, A460, A461, S462, A463, S464, E465, R466, E467, M468, E469, K470, Q471, R472, E473, or V474.

Any of the following amino acids in the root-specific or root-preferred GADs in maize can be changed to stop codons to practice the invention.
Maize (Zea mays) from the DNA translation of NP_001167941 into amino acids 1 to 490:
A458, P459, L460, L461, R462, K463, K464, T465, E466, L467, E468, T469, Q470, or R471.
Maize (Zea mays) from the DNA translation of NP_001130451 into amino acids 1 to 493:
L459, A460, A461, A462, E463, S464, S465, E466, R467, E468, M469, E470, K471, Q472, R473, Q474, or V475.

Those of ordinary skill in the art know that stop codons TAA, TAG or TGA can be inserted as a triplet into a nucleotide sequence to terminate translation of a peptide. A tri-nucleotide, TAA, TAG or TGA, can replace an existing codon or a nucleotide can be inserted in front of an existing amino acid codon to form a stop codon. In addition, the conversion of an existing amino acid codon into a stop codon can be achieved by single or double nucleotide replacements. For example, an AAA (Lys) can be converted to TAA (a stop codon) with the replacement of an A to a T. In addition, an AAA (Lys) can be converted to a stop codon with a double mutation to TGA or TAG. Those of ordinary skill in the art know that the conversion of an amino acid into a stop codon can be achieved by the insertion of a nucleotide prior to the amino acid codon. For example, the conversion of AGT (Ser) to TAG (stop codon) can be achieved with the insertion of a T, before the AGT to form TAGT. Those of ordinary skill in the art know that the conversion of an amino acid into a stop can be achieved by the deletion of one or two nucleotides prior to the amino acid codon or in an amino acid codon. For example, the conversion of CTA (Leu) to a stop codon can be achieved in the following sequence CTAGGG (LeuGly) by the deletion of the C to make TAG (stop codon).

Site-specific mutagenesis or genome editing protocols that are known to those of ordinary skill in the art are utilized in practicing the present invention. Site-specific mutations can be accomplished in a wide variety of ways (60, 61). These editing systems, which are well known to those of ordinary skill in the art, are suitable for use in cells in the present invention and include but are not limited to ZFNs (51, 67, 81), TALENs (64, 70, 71), CRISPR/Cas (54, 72, 82-85) and ODMs (57, 58, 74, 86, 87).

Biochemical, Molecular and Targeted Mutation Techniques

For purposes of promoting an understanding of the principles of the invention, reference will now be made to particular embodiments of the invention and specific language will be used to describe the same. The materials, methods and examples are illustrative only and not limiting. Unless mentioned otherwise, the techniques employed or contemplated herein are standard methodologies well known to one of ordinary skill in the art. Specific terms, while employed below and defined at the end of this section, are used in a descriptive sense only and not for purposes of limitation. The practice of the present invention will employ, unless otherwise indicated, conventional techniques of botany, microbiology, tissue culture, molecular biology, chemistry, biochemistry and recombinant DNA technology, which are within the skill of the art (51, 59-63, 88-94).

Transformation of Host Cells

Transformation of a plant can be accomplished in a wide variety of ways within the scope of a person of ordinary skill in the art. In one embodiment, an ssODN or DNA construct is incorporated into a plant by (i) transforming a cell, tissue or organ from a host plant with the ssODN or DNA construct; (ii) selecting a transformed cell, cell callus, somatic embryo, or seed which contains the ssODN or DNA construct; (iii) regenerating a whole plant from the selected transformed cell, cell callus, somatic embryo, or seed; and (iv) selecting a regenerated whole plant that expresses the mutated polynucleotide. Many methods of transforming a plant, plant tissue or plant cell for the construction of a transformed cell are suitable. Once transformed, these cells can be used to regenerate transgenic plants (95).

In one embodiment of the invention, a transformed host cell may be cultured to produce a transformed plant. In this regard, a transformed plant can be made, for example, by transforming a cell, tissue or organ from a host plant with an inventive ssODN or DNA construct; selecting a transformed cell, cell callus, somatic embryo, or seed which contains the ssODN or DNA construct; regenerating a whole plant from the selected transformed cell, cell callus, somatic embryo, or seed; and selecting a regenerated whole plant that expresses the truncGAD polynucleotide.

The preferred methods for delivery of the mutation to the host cells are particle or micro-projectile bombardment or ballistic particle acceleration (96-99) for the ssODN and CRISPR/Cas9 systems or by agrobacterium-mediated transformation for the CRISPR/Cas9 system (100, 101). In addition, those of ordinary skill in the art know that different plant gene transfer techniques may be used, including electroporation (102-106), microinjection (107, 108), lipofection (109), liposome or spheroplast fusions (110-112), direct gene transfer (113), T-DNA mediated transformation of monocots (114), chemical transfection including CaCl₂ precipitation, polyvinyl alcohol, or poly-L-ornithine (115), silicon carbide whisker methods (116), laser methods (117, 118), sonication methods (119-121), polyethylene glycol methods (122), vacuum infiltration (123) and transbacter (124).

The methods described above may be applied to transform a wide variety of plants, angiosperm and gymnosperm, including decorative or recreational plants or crops, but are particularly useful for treating commercial and ornamental crops. Examples of plants that may be transformed in the present invention include, but are not limited to, Acacia, alfalfa, algae, aneth, apple, apricot, artichoke, arugula, asparagus, avocado, banana, barley, beans, beech, beet, Bermuda grass, bent grass, blackberry, blueberry, Blue grass, broccoli, Brussels sprouts, cabbage, camelina, canola, cantaloupe, carinata, carrot, cassava, cauliflower, celery, cherry, chicory, cilantro, citrus, clementines, coffee, corn, cotton, cucumber, duckweed, Douglas fir, eggplant, endive, escarole, eucalyptus, fennel, fescue, figs, forest trees, garlic, gourd, grape, grapefruit, honey dew, jicama, kiwifruit, lettuce, leeks, lemon, lime, Loblolly pine, maize, mango, melon, mushroom, nectarine, nut, oat, okra, onion, orange, an ornamental plant, papaya, parsley, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, rye grass, seaweed, scallion, sorghum, Southern pine, soybean, spinach, squash, strawberry, sudangrass, sugar beet, sugarcane, sunflower, sweet potato, sweetgum, switchgrass, tangerine, tea, tobacco, tomato, triticale, turf, turnip, a vine, watermelon, wheat, yams, and zucchini.

The invention provides pharmaceutical compositions that comprise extracts of one or more non-transgenic truncGAD plants described above. Plant extracts containing GABA can be used to synthesize or manufacture GABA. GABA can function as an anti-hypertension (125). Extracts from GABA-containing plants may be used in pharmaceutical or medicinal compositions to deliver GABA for use in the treatments (38). Pharmaceutically acceptable vehicles of GABA are tablets, capsules, gel, ointment, film, patch, powder or dissolved in liquid form.

Nutritional Supplements and Feeds

Non-transgenic truncGAD plants containing GABA may be consumed or used to make extracts for nutritional supplements. TruncGAD plant parts that contain GABA may be used for human consumption. The plant parts may include but are not limited to leaves, stalks, stems, tubers, stolons, roots, petioles, cotyledons, seeds, fruits, grain, strover, nuts, flowers, petioles, pollen, buds, or pods. Extracts from truncGAD plants containing GABA may be used as nutritional supplements, as an antioxidant. The extracts may be used in the form of a liquid, powder, capsule or tablet.

Non-transgenic truncGAD plants containing GABA may be used as animal feed or used to make extracts for the supplementation of animal feed. Plant parts of truncGAD plants that contain GABA may be used as animal or fish feed. The plant parts include but are not limited to leaves, stalks, stems, tubers, stolons, roots, petioles, cotyledons, seeds, fruits, grain, strover, nuts, flowers, petioles, buds, pods, or husks. Extracts from truncGAD plants containing GABA may be used as feed supplements in the form of a liquid, powder, capsule or tablet.

Enhancer of Plant Growth or Yield

Non-transgenic truncGAD plants may be used as an enhancer for plant growth or yield. The plant parts include, but are not limited to, leaves, stalks, stems, tubers, stolons, roots, petioles, cotyledons, seeds, fruits, grain, strover, nuts, flowers, petioles, buds, pods, or husks. Extracts from truncGAD plants containing GABA may be used as plant growth enhancers, ripeners or supplemental fertilizers in the form of a liquid, powder, capsule or tablet.

GABA could be purified from the cells or from extracts of the cells or from media from which the cells were grown. The extracted GABA could be used as a food or feed additive, nutrient, pharmaceutical or an enhancer of plant growth or yield.

Methods such as centrifugation, filtration, crystallization, ion exchange, electrodialysis, solvent extraction, decolorization or evaporation to purify or separate chemical compounds from cells or from liquids or media that grew cells are well known to one with ordinary skill in the art. These methods can be used by one with ordinary skill in the art to purify or separate GABA from cells with the invention, or from liquids or media from which cell suspensions or cell cultures containing the invention were grown (126-132).

### Definitions

The term “polynucleotide” refers to a natural or synthetic linear and sequential array of nucleotides and/or nucleosides, including deoxyribonucleic acid, ribonucleic acid, and derivatives thereof. It includes chromosomal DNA, self-replicating plasmids, infectious polymers of DNA or RNA and DNA or RNA that performs a primarily structural role. Unless otherwise indicated, nucleic acids or polynucleotide are written left to right in 5′ to 3′ orientation, Nucleotides are referred to by their commonly accepted single-letter codes. Numeric ranges are inclusive of the numbers defining the range.

The term “plant” includes whole plants, and plant organs, and progeny of same. Plant organs comprise, e.g., shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like). The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

The terms “encoding” and “coding” refer to the process by which a polynucleotide, through the mechanisms of transcription and translation, provides the information to a cell from which a series of amino acids can be assembled into a specific amino acid sequence to produce a functional polypeptide, such as, for example, an active enzyme or ligand binding protein.

The terms “polypeptide,” “peptide,” “protein” and “gene product” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. Amino acids may be referred to by their commonly known three-letter or one-letter symbols. Amino acid sequences are written left to right in amino to carboxy or carboxl orientation, respectively. Numeric ranges are inclusive of the numbers defining the range.

The terms “residue,” “amino acid residue,” and “amino acid” are used interchangeably herein to refer to an amino acid that is incorporated into a protein, polypeptide, or peptide. The amino acid may be a naturally occurring amino acid and may encompass known analogs of natural amino acids that can function in a similar manner as the naturally occurring amino acids.

The terms “glutamate decarboxylase”, “glutamic acid decarboxylase” and “GAD” (EC 4.1.1.15) refers to the protein that catalyzes a decarboxylase reaction which cleaves carbon-carbon bonds and includes, but is not limited to, the following substrate and end-products:

Glutamate=4-aminobutanoate+CO₂

Note: another name for 4-aminobutanoate is gamma-aminobutyric acid (GABA).

The term “mutant”, “mutated” or “mutagenesis” includes reference to a GAD nucleotide sequence that has been modified to change an amino acid codon into a stop codon to make a truncGAD. A mutation in the amino acid codon to a stop codon (TAA, TGA or TAG in the sense strand) could be due to the insertion of one, two or three nucleotides. A mutation in the amino acid codon to a stop codon could be due to the deletion of one or two nucleotides.

The term “root-preferred” refers to a gene that is under the control of a promoter that preferentially initiates transcription of the gene and accumulates the corresponding peptide in root tissue.

The term “root-specific” refers to a gene that is under the control of a promoter that only initiates transcription of the gene and accumulates the corresponding peptide in root tissue.

The term “ODM” refers to synthetic single-stranded oligonucleotides (ssODNs), chimeraplasts, recombinagenic oligonucleobases or single-stranded oligodeoxynucleotide mutational vector (SSOMV) that are a mixture of DNA and RNA and are 41 to 201 nucleotides in length. The nucleotide bases can be modified to increase binding to complementary DNA targets or to protect against nuclease degradation. The ssODNs can be designed to be complementary to either the coding or the non-coding strand of the target nucleotide sequence. When the desired mutation is a substitution of a single base, it is preferred that the nucleotide for the mutation site be a pyrimidine.

The term “vector” includes reference to a nucleic acid used in transfection or transformation of a host cell and into which can be inserted a polynucleotide.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity, and most preferably 100% sequence identity (i.e., complementary) with each other.

The terms “stringent conditions” and “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

The following terms are used to describe the sequence relationships between two or more nucleic acids or polynucleotides or polypeptides: “reference sequence”, “comparison window,” “sequence identity,” “percentage of sequence identity,” and “substantial identity.”

The term “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

The term “comparison window” includes reference to a contiguous and specified segment of a polynucleotide sequence, where the polynucleotide sequence may be compared to a reference sequence and the portion of the polynucleotide sequence in the comparison window may comprise additions or deletions (i.e., gaps) when it is compared to the reference sequence for optimal alignment. The comparison window is usually at least 20 contiguous nucleotides in length, and optionally can be 30, 40, 50, 100 or longer. Those of ordinary skill in the art understand that the inclusion of gaps in a polynucleotide sequence alignment introduces a gap penalty, and it is subtracted from the number of matches. The terms “sequence identity” and “identity” are used in the context of two nucleic acid or polypeptide sequences and include reference to the residues in the two sequences, which are the same when aligned for maximum correspondence over a specified comparison window. When the percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conserved substitutions, the percent sequence identity may be adjusted upwards to correct for the conserved nature of the substitution. Sequences, which differ by such conservative substitutions, are said to have “sequence similarity” or “similarity.” Scoring for a conservative substitution allows for a partial rather than a full mismatch (133), thereby increasing the percentage sequence similarity.

The term “percentage of sequence identity” means the value determined by comparing two optimally aligned sequences over a comparison window, wherein the portion of the polynucleotide sequence in the comparison window may comprise gaps (additions or deletions) when compared to the reference sequence for optimal alignment. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” in the context of a peptide indicates that a peptide comprises a sequence with between 40-100% sequence identity to a reference sequence preferably at least 40% sequence identity, preferably 60% preferably 70%, more preferably 80%, most preferably at least 90% or 95% sequence identity to the reference sequence over a specified comparison window. Preferably, optimal alignment is conducted using the homology alignment algorithm (134). Thus, a peptide is substantially identical to a second peptide, for example, where the two peptides differ only by a conserved substitution. Another indication that amino acid sequences are substantially identical is if two polypeptides immunologically cross-react with the same antibody in a western blot, immunoblot or ELISA assay. In addition, a peptide can be substantially identical to a second peptide when they differ by a non-conservative change if the epitope that the antibody recognizes is substantially identical.

All patents, patent applications, and references cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

### Example 1

### Development of a Non-Transgenic Sugar Beet Plant with a Root-Preferred truncGAD

Mutate the root-preferred XP_010690484 gene in sugar beet to make a non-transgenic plant with a root-preferred truncGAD by conversion of the codon for K471 into a stop codon. Use a ssODN or gRNA that contains the following nucleotide or ribonucleotide as part of the recognition sequence:

SEQ ID NO: 15
GCTTCTAGGAAGtAGACTGCCTTGG

The ssODN or gRNA is made according to techniques known to those of ordinary skill in the art. The ssODN is preferably delivered into a sugar beet plant cell by particle bombardment. The gRNA is used in a CRISPR/Cas9 system and preferably delivered into a sugar beet plant cell via agrobacterium-mediated transformation or particle bombardment. Regenerated plants that contain the truncGAD at position 471 due to the insertion of a stop codon are identified by PCR (FIG. 6) using the following sets of primers:

Set one:
SEQ ID NO: 16
GGATGGTGAAAATCAAGCTTCTAGGAAGT
and
SEQ ID NO: 17
CCATGATCATACTCAACTAAACCTCACTCCC
Set two:
SEQ ID NO: 18
TTTGGTTGGATTGTACCAGCTTACACCATG
and
SEQ ID NO: 19
GACCTCCATTTGCATCTCCAAGGCAGTCTA

### Example 2

### Effects of a Root-Preferred truncGAD in Sugar Beet on GABA/Glu Ratios

Sugar beet plants (PCR-positive truncGAD plants and PCR-negative null plants) were grown in 16 hr-day/8 hr-night conditions in a greenhouse for 180 days. Roots were then harvested and processed in a juicer. The supernatant was analyzed for free amino acids and GABA using HPLC as described by Renault et al. (135). The non-transgenic sugar beet plant with a root-preferred truncGAD had a higher GABA/Glu ratio than null sugar beet plants that did not contain the mutation. The truncGAD sugar beet plant had a GABA/Glu ratio of 85.6, which was 12% higher than the average GABA/Glu ratio of the three nulls, mean=7.4, 95% confidence interval [5.92, 8.94].

### Example 3

### Effects of a Root-Preferred truncGAD in Sugar Beet on °Bx

Sugar beet plants (PCR-positive truncGAD plants and PCR-negative null plants) were grown in 16 hr-day/8 hr-night conditions in a greenhouse for 180 days. Roots were then harvested and processed in a juicer. The supernatant from their roots was analyzed for total sugar content using a refractometer to obtain a refractive index (degrees Brix [°Bx]). The non-transgenic sugar beet plant with a root-preferred truncGAD had higher °Bx than the null sugar beet plants that did not contain the mutation. The truncGAD sugar beet plant had a °Bx value of 24.8, which was 17% higher than the average °Bx value of the three nulls, mean=21.1, 95% confidence interval [19.4, 22.9]. Note, °Bx measures are highly correlated (r=0.96) (136) with measures of soluble sugars obtained using an enzymatic method and spectrophotometry (136, 137).

### Example 4

### Development of Root-Preferred or Root-Specific truncGAD Mutants

The preferred amino acid targets that can be converted to stop codons are presented below, however any of the amino acid codons identified in Table 3 can be mutated into stop codons to make a truncGAD using similarly design ODMs or gRNAs. In the examples presented below the top sequence is the native gene target and the bottom sequence is the sequence for the ssODN or gRNA to make the mutation. Lower case letters represent the mutation in the ssODN or gRNA. Bold italicized letters in the top sequence represent deletions in the mutated sequence. Gaps are represented with a dash (-). The in-frame stop codon is underlined in the bottom sequences. Note, if a gRNA is used to make the mutation only 20 nucleotides are required and the mutations should be located toward the 5′ end (greater than 10 nucleotides from the 3′ end) of the gRNA. This is achieved by removing nucleotides from the 5′ end of the bottom sequence. For example: if SEQ ID NO: 15 where used in a gRNA the first 5 nucleotides (at the 5′) end would be removed to use TAGGAAGtAGACTGCCTTGG as the gRNA.

For the formation of a truncGAD in sugar beet XP_010690484:

Convert the codon for K471 into a stop codon by replacement of an A with a T,

SEQ ID NO: 37
GCTTCTAGGAAGAAGACTGCCTTGG
SEQ ID NO: 15
GCTTCTAGGAAGtAGACTGCCTTGG

or convert the codon for N482 into a stop codon by insertion of a T,

SEQ ID NO: 38
ATGGAGGTCTGC-AATGTTTGGAAG
SEQ ID NO: 20
ATGGAGGTCTGCtAATGTTTGGAAG

or convert the codon for A460 into a stop codon by replacement of a GCT with a TAG (could also use TAA or TGA).

SEQ ID NO: 39
AATGTTCCACACGCTGTGGATGGTGAAA
SEQ ID NO: 21
AATGTTCCACACtagGCTGTGGATGGTG

For the formation of a truncGAD in sugar beet XP_010687254:

Convert the codon for K476 into a ston codon by replacement of an A with a T,

SEQ ID NO: 40
GCTGTTATCAAAAAAGATGTGGTAG
SEQ ID NO: 22
GCTGTTATCAAAtAAGATGTGGTAG

or convert the codon for K487 into a stop codon by insertion of a T,

SEQ ID NO: 41
AAGGACATAACC_AAGTACTGGAAA
SEQ ID NO: 23
AAGGACATAACCtAAGTACTGGAAA

or convert the codon for K464 into a stop codon by insertion of a T.

SEQ ID NO: 42
GTCACCCTTGAC-AAGGATGAAACA
SEQ ID NO: 24
GTCACCCTTGACtAAGGATGAAACA

For the formation of a truncGAD in soybean Glyma.11g213000 or NP_001276214:

Convert the codon for K472 into a ston codon by replacement of an A with a T,

SEQ ID NO: 43
GTAGTGGTTGCTAAGAAGAGTGCTA
SEQ ID NO: 25
GTAGTGGTTGCTtAGAAGAGTGCTA

or convert the codon for FASO into a ston codon by replacement of a G with a T,

SEQ ID NO: 44
GAGACTCAGAGGGAAATCACTGCCA
SEQ ID NO: 26
GAGACTCAGAGGtAAATCACTGCCA

or convert the codon for E462 into a stop codon by replacement of a G with a T.

SEQ ID NO: 45
ACAGTCACTGCTGAAGAAAATGGCA
SEQ ID NO: 27
ACAGTCACTGCTtAAGAAAATGGCA

For the formation of a truncGAD in in rice LOC 0s03g51080 or XP_015631723:

Convert the codon for K470 into a stop codon by replacement of an A with a T,

SEQ ID NO: 46
AGGGAGATGGAGAAGCAGCGCGAGG
SEQ ID NO: 28
AGGGAGATGGAGtAGCAGCGCGAGG

or convert the codon for V474 into a stop codon with the deletion of a G,

SEQ ID NO: 47
AAGCAGCGCGAG [symbol] TGATCTCCCTCTG
SEQ ID NO: 29
AAGCAGCGCGAG-TGATCTCCCTCTG

or convert the codon for A459 into a stop codon by replacement of a GCC with a TAG (could also use TAA or TGA).

SEQ ID NO: 48
GGCGGCGACGCCGCCGCCGCGTCGGCG
SEQ ID NO: 30
GGCGGCGACGCCtagGCCGCGTCGGCG

For the formation of a truncGAD in maize GRMZM2G098875_P02 or NP_001167941:

Convert the codon for K464 into a stop codon by replacement of an A with a T,

SEQ ID NO: 49
CTGCTCAGGAAAAAGACGGAGCTGG
SEQ ID NO: 31
CTGCTCAGGAAAtAGACGGAGCTGG

or convert the codon for R471 into a stop codon by insertion of a T,

SEQ ID NO: 50
CTGGAGACGCAG-AGGTCGGTCACGG
SEQ ID NO: 32
CTGGAGACGCAGtAGGTCGGTCACGG

or convert the codon for A458 into a stop codon by replacement of a GCG with a TAG (could also use TAA or TGA).

SEQ ID NO: 51
CCTCCCCCGGCGGCGCCGCTGCTCAGG
SEQ ID NO: 33
CCTCCCCCGGCGtagCCGCTGCTCAGG

For the formation of a truncGAD in maize GRMZM5G826838_P01 or NP_001130451:

Convert the codon for E466 into a stop codon by replacement of an G with a T,

SEQ ID NO: 52
GCCGAGTCCAGCGAGAGGGAGATGG
SEQ ID NO: 34
GCCGAGTCCAGCtAGAGGGAGATGG

or convert the codon for V475 into a stop codon with the deletion of a G,

SEQ ID NO: 53
AAGCAGCGCCAG [symbol] TGATCTCGCTCTG
SEQ ID NO: 35
AAGCAGCGCCAG-tgaTCTCGCTCTG

or convert the codon for L459 into a stop codon by replacement of a CTC with a TAG (could also use TAA or TGA).

SEQ ID NO: 54
GACCTAGCCGCGCTCGCCGCGGCCGAGTC
SEQ ID NO: 36
GACCTAGCCGCGtagGCCGCGGCCGAGTC

### REFERENCES

1. Bouché N, Fait A, Bouchez D, Moller S G, & Fromm H (2003) Mitochondrial succinic-semialdehyde dehydrogenase of the gamma-aminobutyrate shunt is required to restrict levels of reactive oxygen intermediates in plants. Proc Natl Acad Sci USA 100(11):6843-6848.
2. Palanivelu R, Brass L, Edlund A F, & Preuss D (2003) Pollen tube growth and guidance is regulated by POP2, an Arabidopsis gene that controls GABA levels. Cell 114(1):47-59.
3. Streeter J G & Thompson J F (1972) In vivo and in vitro studies on γ-aminobutyric acid metabolism with the radish plant (Raphanus sativus, L.). Plant Physiol 49(4):579.
4. Wallace W (1984) Rapid accumulation of γ-aminobutyric acid and alanine in soybean leaves in response to an abrupt transfer to lower temperature, darkness or mechanical manipulation. Plant Physiol 75:170-175.
5. Rhodes D, Handa S, & Bressan R A (1986) Metabolic changes associated with adaptation of plant cells to water stress. Plant Physiol 82(4):890.
6. Mayer R R, Cherry J H, & Rhodes D (1990) Effects of heat shock on amino Acid metabolism of cowpea cells. Plant Physiol 94(2):796-810.
7. Bown A W & Shelp B J (1997) The metabolism and functions of [gamma]-aminobutyric acid. Plant Physiol 115(1):1-5.
8. Breitkreuz K E, Shelp B J, Fischer W N, Schwacke R, & Rentsch D (1999) Identification and characterization of GABA, proline and quaternary ammonium compound transporters from Arabidopsis thaliana. FEBS Lett 450(3):280-284.
9. Snedden W A & Fromm H (1999) Regulation of the γ-aminobutyrate-synthesizing enzyme, glutamate decarboxylase, by calcium-calmodulin: a mechanism forrapid activation in response to stress. Plant Responses to Environmental Stresses, ed Lerner HR (Marcel Dekker, New York), pp 549-576.
10. Kinnersley A M & Turano F J (2000) Gamma Aminobutyric Acid (GABA) and Plant Responses to Stress. Crit Rev Plant Sci 19:479-509.
11. Kathiresan A, Miranda J, Chinnappa C C, & Reid D M (1998) g-aminobutyric acid promotes stem elongation in Stellaria longipes: the role of ethylene. Plant Growth Reg 26:131-137.
12. Kinnersley A M & Lin F (2000) Receptor modifiers indicate that GABA is a potential modulator of ion transport in plants. Plant Growth Reg 9:137-146.
13. Li W, et al. (2016) Exogenous γ-aminobutyric acid (GABA) application improved early growth, net photosynthesis, and associated physio-biochemical events in maize. Front Plant Sci 7:919.
14. Song H, Xu X, Wang H, Wang H, & Tao Y (2010) Exogenous γ-aminobutyric acid alleviates oxidative damage caused by aluminium and proton stresses on barley seedlings. J Sci Food Agric 90(9):1410-1416.
15. Wang Y, et al. (2017) γ-Aminobutyric acid imparts partial protection from salt stress injury to maize seedlings by improving photosynthesis and upregulating osmoprotectants and antioxidants. Sci Rep 7:43609.
16. Schirra M, D'Aquino S, Cabras P, & Angioni A (2011) Control of postharvest diseases of fruit by heat and fungicides: efficacy, residue levels, and residue persistence. A review. J Agric Food Chem 59:8531-8542.
17. Yang A, Cao S, Yang Z, Cai Y, & Zheng Y (2011) γ-Aminobutyric acid treatment reduces chilling injury and activates the defence response of peach fruit. Food Chem 129(4):1619-1622.
18. Aghdam M S, Naderi R, Jannatizadeh A, Sarcheshmeh M A A, & Babalar M (2016) Enhancement of postharvest chilling tolerance of anthurium cut flowers by γ-aminobutyric acid (GABA) treatments. Sci Hortic 198:52-60.
19. Malekzadeh P, Khara J, & Heydari R (2014) Alleviating effects of exogenous Gamma-aminobutiric acid on tomato seedling under chilling stress. Physiol Mol Biol Plants 20(1):133-137.
20. Krishnan S, Laskowski K, Shukla V, & Merewitz E B (2013) Mitigation of drought stress damage by exogenous application of a non-protein amino acid γ-aminobutyric acid on perennial ryegrass. J Am Soc Hortic Sci 138:358-366.
21. Yong B, et al. (2017) Exogenous application of GABA improves PEG-induced drought tolerance positively associated with GABA-shunt, polyamines, and proline metabolism in white clover. Front Physiol 8(1107).
22. Vijayakumari K & Puthur J T (2016) γ-Aminobutyric acid (GABA) priming enhances the osmotic stress tolerance in Piper nigrum Linn. plants subjected to PEG-induced stress. Plant Growth Regul 78:57-67.
23. Li Z, Yu J, Peng Y, & Huang B (2016) Metabolic pathways regulated by γ-aminobutyric acid (GABA) contributing to heat tolerance in creeping bentgrass (Agrostis stolonifera). Sci Rep 6:30338.
24. Nayyar H, Kaur R, & Kaur S (2014) γ-aminobutyric acid (GABA) imparts partial protection from heat stress injury to rice seedlings by improving leaf turgor and upregulating osmoprotectants and antioxidants. J Plant Growth Regul 33:408.
25. Mahmud J, et al. (2017) γ-aminobutyric acid (GABA) confers chromium stress tolerance in Brassica juncea L. by modulating the antioxidant defense and glyoxalase systems. Ecotoxicol 26:675.
26. Li M F, Guo S J, Yang X H, Meng Q W, & Wei XJ (2016) Exogenous gamma-aminobutyric acid increases salt tolerance of wheat by improving photosynthesis and enhancing activities of antioxidant enzymes. Biol Plant 60:123-131.
27. McLean M D, et al. (2003) Overexpression of glutamate decarboxylase in transgenic tobacco plants confers resistance to the northern root-knot nematode. Mol Breed 11:277-285.
28. Flores H E & Filner P (1985) Polyamine catabolism in higher plants: Characterization of pyrroline dehydrogenase. Plant Growth Regul 3:277-291.
29. Matsuda H & Suzuki Y (1984) γ-guanidinobutyraldehyde dehydrogenase of Vicia faba leaves. Plant Physiol 76(3): 654-657.
30. Breitkreuz K E & Shelp B J (1995) Subcellular Compartmentation of the 4-Aminobutyrate Shunt in Protoplasts from Developing Soybean Cotyledons. Plant Physiol 108(1):99-103.
31. Li R, et al. (2018) Multiplexed CRISPR/Cas9-mediated metabolic engineering of γ-aminobutyric acid levels in Solanum lycopersicum. Plant Biotechnol 16(2):415-427.
32. MacGregor K B (2003) Overexpression of glutamate decarboxylase in transgenic tobacco plants deters feeding by phytophagous insect larvae. J Chem Ecol 29:2177-2182.
33. Snedden W A, Arazi T, Fromm H, & Shelp B J (1995) Calcium/Calmodulin activation of soybean glutamate decarboxylase. Plant Physiol 108:543-549.
34. Baum G, Chen Y, Arazi T, Takatsuji H, & Fromm H (1993) A plant glutamate decarboxylase containing a calmodulin binding domain. Cloning, sequence, and functional analysis. J Biol Chem 268:19610-19617.
35. Baum G, et al. (1996) Calmodulin binding to glutamate decarboxylase is required for regulation of glutamate and GABA metabolism and normal development in plants. Embo J 15(12):2988-2996.
36. Akama K, Akihiro T, Kitagawa M, & Takaiwa F (2001) Rice (Oryza sativa) contains a novel isoform of glutamate decarboxylase that lacks an authentic calmodulin-binding domain at the C-terminus. Biochim Biophys Acta 1522(3):143-150.
37. Akama K & Takaiwa F (2007) C-Terminal extension of rice glutamate decarboxylase (OsGAD2) functions as an autoinhibitory domain and overexpression of a truncated mutant results in the accumulation of extremely high levels of GABA in plant cells. J Exp Bot 58:2699-2707.
38. Akama K, et al. (2009) Seed-specific expression of truncated OsGAD2 produces GABA-enriched rice grains that influence a decrease in blood pressure in spontaneously hypertensive rats. Transgenic Res 18(6):865-876.
39. Nonaka S, Arai C, Takayama M, Matsukura C, & Ezura H (2017) Efficient increase of γ-aminobutyric acid (GABA) content in tomato fruits by targeted mutagenesis. Sci Rep 7(1):7057.
40. Akama K & Takaiwa F (2007) C-terminal extension of rice glutamate decarboxylase (OsGAD2) functions as an autoinhibitory domain and overexpression of a truncated mutant results in the accumulation of extremely high levels of GABA in plant cells. J Exp Bot 58(10):2699-2707.
41. Fait A, et al. (2011) Targeted enhancement of glutamate-to-γ-aminobutyrate conversion in Arabidopsis seeds affects carbon-nitrogen balance and storage reserves in a development-dependent manner. Plant Physiol 157(3):1026.
42. Turano FJ & Fang TK (1998) Characterization of two glutamate decarboxylase cDNA clones from Arabidopsis. Plant Physiol 117(4):1411-1421.
43. Bouche N, Fait A, Zik M, & Fromm H (2004) The root-specific glutamate decarboxylase (GAD1) is essential for sustaining GABA levels in Arabidopsis. Plant Mol Biol 55(3):315-325.
44. Takayama M, Matsukura C, Ariizumi T, & Ezura H (2017) Activating glutamate decarboxylase activity by removing the autoinhibitory domain leads to hyper gamma-aminobutyric acid (GABA) accumulation in tomato fruit. Plant cell reports 36(1):103-116.
45. Turano F & Turano K (2012) U.S. Pat. No. 8,106,261.
46. Turano F & Turano K (2013) U.S. Pat. No. 8,581,040.
47. Turano F & Turano K (2013) U.S. Pat. No. 8,581,041.
48. Turano F, Turano K, Carlson P S, & Kinnersley A M (2018) U.S. Pat. No. 9,267,148.
49. Townsend J A, et al. (2009) High-frequency modification of plant genes using engineered zinc-finger nucleases. Nature 459:442.
50. Carroll D (2011) Genome engineering with zinc-finger nucleases. Genetics 188(4):773-782.
51. Carlson D F, Fahrenkrug S C, & Hackett P B (2012) Targeting DNA with fingers and TALENs. Molecular therapy. Nucleic acids 1(1):e3.
52. Gupta A, et al. (2012) An optimized two-finger archive for ZFN-mediated gene targeting. Nat Methods 9(6):588-590.
53. Joung J K & Sander J D (2013) TALENs: a widely applicable technology for targeted genome editing. Nat Rev Mol Cell Biol 14(1):49-55.
54. Li J-F, et al. (2013) Multiplex and homologous recombination-mediated genome editing in Arabidopsis and Nicotiana benthamiana using guide RNA and Cas9. Nature Biotechnol 31:688-691.
55. Shan Q, et al. (2013) Targeted genome modification of crop plants using a CRISPR-Cas system. Nature Biotechnol 31:686-688.
56. Nekrasov V, Staskawicz B, Weigel D, Jones J D G, & Kamoun S (2013) Targeted mutagenesis in the model plant Nicotiana benthamiana using Cas9 RNA-guided endonuclease. Nature Biotechnol 31:691-693.
57. Gocal G F W, Knuth M E, & Beetham R (2012) U.S. Pat. No. 8,268,622.
58. Beetham P R, Kipp P B, Sawycky X L, Arntzen C J, & May G D (1999) A tool for functional plant genomics: Chimeric RNA/DNA oligonucleotides cause in vivo gene-specific mutations. Proc Natl Acad Sci USA 96(15):8774.
59. Sauer N J, et al. (2016) Oligonucleotide-mediated genome editing provides precision and function to engineered nucleases and antibiotics in plants. Plant Physiol 170(4):1917.
60. Weeks D P, Spalding M H, & Yang B (2016) Use of designer nucleases for targeted gene and genome editing in plants. Plant Biotechnol 14(2):483-495.
61. Songstad D D, Petolino J F, Voytas D F, & Reichert N A (2017) Genome editing of plants. Crit Rev Plant Sci 36(1):1-23.
62. Kamburova V S, et al. (2017) Genome editing in plants: An overview of tools and applications. Int J Agronomy 2017:1-15.
63. Mohanta T K, Bashir T, Hashem A, Abd Allah E F, & Bae H (2017) Genome editing tools in plants. Genes 8(12):399.
64. Malzahn A, Lowder L, & Qi Y (2017) Plant genome editing with TALEN and CRISPR. Cell Biosci 7:21.
65. Sauer N J, et al. (2016) Oligonucleotide-mediated genome editing provides precision and function to engineered nucleases and antibiotics in plants. Plant Physiol 170(4):1917-1928.
66. Urnov F D, et al. (2005) Highly efficient endogenous human gene correction using designed zinc-finger nucleases. Nature 435:646-651.
67. Shukla V K, et al. (2009) Precise genome modification in the crop species Zea mays using zinc-finger nucleases. 459:437-441.
68. Puchta H (2005) The repair of double-strand breaks in plants: mechanisms and consequences for genome evolution. J Exp Bot 56(409):1-14.
69. Christian M, et al. (2010) Targeting DNA double-strand breaks with TAL effector nucleases. Genetics 186(2):757-761.
70. Zhang Y, et al. (2013) Transcription activator-like effector nucleases enable efficient plant genome engineering. Plant Physiol 161(1):20-27.
71. Li T, Liu B, Spalding M H, Weeks D P, & Yang B (2012) High-efficiency TALEN-based gene editing produces disease-resistant rice. Nature Biotechnol 30:390.
72. Liu X, Wu S, Xu J, Sui C, & Wei J (2017) Application of CRISPR/Cas9 in plant biology. Acta Pharmaceutica Sinica B 7(3):292-302.
73. Makarova K S, et al. (2011) Evolution and classification of the CRISPR-Cas systems. Nature Rev Microbiol 9:467-477.
74. Papaioannou I, Simons J P, & Owen J S (2012) Oligonucleotide-directed gene-editing technology: mechanisms and future prospects. Expert Opin Biol Ther 12(3):329-342.
75. Belfort M & Bonocora R P (2014) Homing endonucleases: From genetic anomalies to programmable genomic clippers Methods Mol Biol 1123:1-26.
76. Arnould S, et al. (2006) Engineering of large numbers of highly specific homing endonucleases that induce recombination on novel DNA targets. J Mol Biol 355(3):443-458.
77. Altschul S F, et al. (1997) Gapped BLAST and PSI-BLAST: a new generation of protein database search programs. Nucleic Acids Res 25:3389-3402.
78. Wootton J C & Federhen S (1993) Statistics of local complexity in amino acid sequences and sequence databases. Comput Chem 17:149-163.
79. Wootton J C & Federhen S (1996) Analysis of compositionally biased regions in sequence databases. Methods Enzymol 266:554-571.
80. Claverie J-M & States DJ (1993) Information enhancement methods for large scale sequence analysis. Comput Chem 17:191-201.
81. Weinthal D, Tovkach A, Zeevi V, & Tzfira T (2010) Genome editing in plant cells by zinc finger nucleases. Trends Plant Sci 15(6):308-321.
82. Gaj T, Gersbach C A, & Barbas C F, 3rd (2013) ZFN, TALEN, and CRISPR/Cas-based methods for genome engineering. Trends Biotechnol 31(7):397-405.
83. Sprink T, Metje J, & Hartung F (2015) Plant genome editing by novel tools: TALEN and other sequence specific nucleases. Curr Opin Biotechnol 32:47-53.
84. Bortesi L & Fischer R (2015) The CRISPR/Cas9 system for plant genome editing and beyond. Biotechnol Adv 33(1):41-52.
85. Kumar V & Jain M (2015) The CRISPR-Cas system for plant genome editing: advances and opportunities. J Exp Bot 66(1):47-57.
86. Zhu T, et al. (1999) Targeted manipulation of maize genes in vivo using chimeric RNA/DNA oligonucleotides. Proc Natl Acad Sci USA 96(15):8768.
87. Arntzen C J, Kipp P B, Kumar R, & May G D (2006) U.S. Pat. No. 7,094,606.
88. Langenheim J H & Thimann K V (1982) Botany: Plant Biology and its Relation to Human Affairs (John Wiley & Sons Inc., New York).
89. Vasil I K (1984) Cell Culture and Somatic Cell Genetics of Plants: Laboratory Procedures and Their Applications (Academic Press, Orlando).
90. Stanier R, Ingrahm J, Wheelis M, & Painter P (1986) The Microbial World (Prentice-Hall, New Jersey).
91. Dhringra O D & Sinclair J B (1985) Basic plant pathology methods (CRC Press, Boca Raton, Fla.).
92. Maniatis T, Fritsch E F, & Sambrook J (1985) Molecular Cloning: A Laboratory Manual: DNA Cloning (Cold Spring Harbor, New York).
93. Hames D D & Higgins S J (1984) Nucleic Acid Hybridization: A Practical Approach (IRL Press, Washington D.C.).
94. Watson J D, Gilman M, Witowski J, & Zoller M (1992) Recombinant DNA (Scientific American Books, New York).
95. Shahin E A (1985) Totipotency of tomato protoplasts. Theor Appl Genet 69:235-240.
96. Klein T M, et al. (1988) Transfer of foreign genes into intact maize cells with high-velocity microprojectiles. Proc Natl Acad Sci USA 85(12):4305-4309.
97. Klein T M, Gradziel T, Fromm M E, & Sanford J C (1988) Factors influencing gene delivery into Zea mays cells by high-velocity microprojectiles. Biotechnol 6:559-563.
98. McCabe D E, Swain W F, Martinell B J, & Christou P (1988) Stable transformation of soybean (Glycine max) by particle acceleration. Biotechnol 6:923-926.
99. Sanford J C, Smith F D, & Rushell J A (1993) Optimizing the biolistic process for different biological application. The Methods in Enzymology, ed Wu R (Academic Press, Orlando), Vol 217, pp 483-509.
100. Rogers S G, Horsch, R. B., and Fraley, R. T. 1986. Gene transfer in plants: Production of transformed plants using Ti-plasmid vectors. (1986) Gene transfer in plants: Production of transformed plants using Ti-plasmid vectors. Methods Enzymol 118:627-640.
101. Bevan M W & Chilton M-D (1982) T-DNA of the Agrobacterium Ti and Ri plasmids. Annu Rev Genet 16:357-384.
102. Fromm M, Taylor LP, & V. W (1985) Expression of genes transferred into monocot and dicot plant cells by electroporation. Proc Natl Acad Sci USA 82:5824-5828.
103. Fromm M E, Taylor L P, & Walbot V (1986) Stable transformation of maize after gene transfer by electroporation. Nature 319(6056):791-793.
104. Riggs C D & Bates G W (1986) Stable transformation of tobacco by electroporation: evidence for plasmid concatenation. Proc Natl Acad Sci USA 83(15):5602-5606.
105. D'Halluin K, Bonne E, Bossut M, De Beuckeleer M, & Leemans J (1992) Transgenic maize plants by tissue electroporation. The Plant cell 4:1495-1505.
106. Laursen C M, Krzyzek R A, Flick C E, Anderson P C, & Spencer T M (1994) Production of fertile transgenic maize by electroporation of suspension culture cells Plant Mol Biol 24:51-61
107. Crossway A, et al. (1986) Integration of foreign DNA following microinjection of tobacco mesophyll protoplasts. Mol Gen Genet 202:179-185.
108. Griesbach R J (1983) Protoplast microinjection. Plant Mol Biol Rep 1:32-37.
109. Sporlein B & Koop H-U (1991) Lipofectin: direct gene transfer to higher plants using cationic liposomes. Theor Appl Genet 83:1-5.
110. Ohgawara T, Uchimiya H, & Harada H (1983) Uptake of liposome-encapsulated plasmid DNA by plant protoplasts and molecular fate of foreign DNA Protoplasma 116:145-148.
111. Deshayes A, Herrera-Estrella L, & Caboche M (1985) Liposome-mediated transformation of tobacco mesophyll protoplasts by an Escherichia coli plasmid. EMBO 4(11):2731-2737.
112. Christou P, Murphy J E, & Swain W F (1987) Stable transformation of soybean by electroporation and root formation from transformed callus. Proc Natl Acad Sci USA 84(12):3962-3966.
113. Paszkowski J, et al. (1984) Direct gene transfer to plants. Embo J 3(12):2717-2722.
114. Hooykaas-Van Slogteren G M, Hooykaas P J, & Schilperoort R A (1984) Expression of Ti plasmid genes in monocotyledonous plants infected with Agrobacterium tumefaciens. Nature 311:763-764.
115. Freeman J P, et al. (1984) A Comparison of Methods for Plasmid Delivery into Plant Protoplasts. Plant Cell Physiol 25:1353-1365.
116. Frame B R, et al. (1994) Production of fertile transgenic maize plants by silicon carbide whisker-mediated transformation. Plant J 6:941-948.
117. Guo Y, Liang H, & Berns M W (1995) Laser-mediated gene transfer in rice. Physiol Plantarum 93:19-24.
118. Badr Y A, Kereim M A, Yehia M A, Fouad O O, & Bahieldin A (2005) Production of fertile transgenic wheat plants by laser micropuncture. Photochem Photobiol Sci 4:803-807.
119. Bao S, Thrall B D, & Miller D L (1997) Transfection of a reporter plasmid into cultured cells by sonoporation in vitro. Ultrasound Med Biol 23:953-959.
120. Finer K R & Finer J J (2000) Use of Agrobacterium expressing green fluorescent protein to evaluate colonization of sonication-assisted Agrobacterium-mediated transformation-treated soybean cotyledons. Lett Appl Microbiol 30(5):406-410.
121. Amoah B K, Wu H, Sparks C, & Jones H D (2001) Factors influencing Agrobacterium-mediated transient expression of uidA in wheat inflorescence tissue. J Exp Bol 52(358):1135-1142.
122. Krens F A, Molendijk L, Wullems G J, & Schilperoort R A (1982) In Vitro transformation of plant protoplasts with Ti-plasmid DNA. Nature 296:72-74.
123. Bechtold N & Pelletier G (1998) In planta Agrobacterium-mediated transformation of adult Arabidopsis thaliana plants by vacuum infiltration. Methods Mol Biol 82:259-266.
124. Broothaerts W, et al. (2005) Gene transfer to plants by diverse species of bacteria. Nature 433:629-633.
125. Ma P, Li T, Ji F, Wang H, & Pang J (2015) Effect of GABA on blood pressure and blood dynamics of anesthetic rats. Int J Clin Exp Med 8(8):14296-14302.
126. Höler A, et al. (1998) U.S. Pat. No. 5,840,358.
127. Oka T (1999) Amino acids, production processes. Encyclopedia of Bioprocess Technology: Fermentation, Biocatalysis, and Bioseparation, eds Flickinger M C & Drew S W (Wiley, London).
128. Lee I, Lee K, Namgoong K, & Lee Y-S (2002) The use of ion exclusion chromatography as approved to the normal ion exchange chromatography to achieve a more efficient lysine recovery from fermentation broth. Enzyme Micro Technol 30(6):798-803.
129. Binder M & Uffmann K-E (2002) U.S. Pat. No. 6,465,025.
130. Hermann T (2003) Industrial production of amino acids by coryneform bacteria. J Biotechnol 104(1-3):155-172.
131. Ikeda M (2003) Amino acid production processes. Adv Biochem Eng/Biotechnol 79:1-35.
132. Leuchtenberger W, Huthmacher K, & Drauz K (2005) Biotechnological production of amino acids and derivatives: current status and prospects. Appl Microbiol Biotechnol 69(1):1-8.
133. Myers E W & Miller W (1988) Optimal alignments in linear-space. CABIOS 4:11-17.
134. Needleman SB & Wunsch CD (1970) A general method applicable to the search for similarities in the amino acid sequence of two proteins. J Mol Biol 48:443-453.
135. Renault H, et al. (2010) The Arabidopsis pop2-1 mutant reveals the involvement of GABA transaminase in salt stress tolerance. BMC Plant Biology 10:20-35.
136. Okamura M, Hashida Y, Hirose T, Ohsugi R, & Aoki N (2016) A simple method for squeezing juice from rice stems and its use in the high-throughput analysis of sugar content in rice stems. Plant Prod Sci 19:309-314.
137. Stitt M, Lilley R M, Gerhardt R, & Heldt H W (1989) [32] Metabolite levels in specific cells and subcellular compartments of plant leaves. Meth Enzymol, Vol 174, pp 518-552.

All patents, patent applications, and references cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A non-transgenic plant cell comprising a truncated GAD (truncGAD) that expresses a truncGAD polypeptide. The truncGAD gene is derived by a mutation in a GAD gene at one of the following amino acid positions to make a stop codon, the positions include A460, V461, D462, G463, E464, N465, Q466, A467, S468, R469, K470, K471, T472, A473, L474, E475, M476, Q477, M478, E479, V480, C481, or N482 in Beta vulgaris (sugar beet) GAD peptide (SEQ ID NO:3) or at an analogous amino acid position in a plant GAD homolog, ortholog or paralog wherein any one of the amino acid codons in those positions are stop codons and a truncGAD peptide is made.

2. A non-transgenic plant cell regenerated into a plant comprising a truncGAD gene.

3. A non-transgenic seed having in its genome endogenous DNA encoding a truncGAD from the regenerated plant of claim 2.

4. A non-transgenic plant grown from the non-transgenic seed of claim 3.

5. The non-transgenic plant of claim 4 which is selected from the group consisting of acacia, alfalfa, algae, aneth, apple, apricot, artichoke, arugula, asparagus, avocado, banana, barley, beans, beech, beet, Bermuda grass, bent grass, blackberry, blueberry, Blue grass, broccoli, Brussels sprouts, cabbage, camelina, canola, cantaloupe, carinata, carrot, cassava, cauliflower, celery, cherry, chicory, cilantro, citrus, clementines, coffee, corn, cotton, cucumber, duckweed, Douglas fir, eggplant, endive, escarole, eucalyptus, fennel, fescue, figs, forest trees, garlic, gourd, grape, grapefruit, honey dew, jicama, kiwifruit, lettuce, leeks, lemon, lime, Loblolly pine, maize, mango, melon, mushroom, nectarine, nut, oat, okra, onion, orange, an ornamental plant, papaya, parsley, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, rye grass, seaweed, scallion, sorghum, Southern pine, soybean, spinach, squash, strawberry, sudangrass, sugar beet, sugarcane, sunflower, sweet potato, sweetgum, switchgrass, tangerine, tea, tobacco, tomato, triticale, turf, turnip, a vine, watermelon, wheat, yams, and zucchini.

6. A method of producing a crop of non-transgenic plants, comprising multiplying or breeding plant material to obtain a crop of non-transgenic plants from the non-transgenic seed of claim 3.

7. A non-transgenic plant of claim 4 comprising a truncGAD gene expressed in the root.

8. A non-transgenic plant wherein truncGAD enzymatic activity increases the production of GABA or increases the GABA/Glu ratio in cells of the non-transgenic plant.

9. A non-transgenic plant having in its genome a truncGAD, wherein the plant has increased growth, yield, biomass, sugars or tolerance to biotic (pests, pathogens, bacteria, microbes, viruses, viroids, microorganisms, invertebrates, insects, nematodes, or vertebrates) or abiotic (changes in osmotic conditions, oxidative damage, drought, salt, cold, freezing, or heat) stresses.

10. The non-transgenic plant cell of claim 2 regenerated into a plant is sugar beet.

11. A pharmaceutical composition comprising an extract of the non-transgenic plant of claim 4, wherein the extract comprises GABA.

12. A nutritional supplement comprising an extract of the non-transgenic plant of claim 4.

13. An animal feed supplement comprising the non-transgenic plant of claim 4.

14. An animal feed supplement comprising the non-transgenic seed of claim 3.


SEQUENCE LISTING (54 sequences)

SEQ ID NO: 1 - protein, 500 aa, Petunia x hybrida
MVLSKTVSQSDVSIHSTFASRYVRTSLPRFKMPDNSIPKEAAYQIINDELMLDGNPRLNLASFVTTWMEPECDKLMMDSINKNYVDMDEYPVTTELQNRCVNMIAHLFNAPLEDGETAVGVGTVGSSEAIMLAGLAFKRKWQNKMKAQGKPCDKPNIVTGANVQVCWEKFARYFEVELKEVKLSEGYYVMDPEKAVEMVDENTICVAAILGSTLNGEFEDVKRLNDLLVEKNKETGWDTPIHVDAASGGFIAPFIYPELEWDFRLPLVKSINVSGHKYGLVYAGIGWVVWRNKDDLPDELIFHINYLGADQPTFTLNFSKGSSQVIAQYYQLIRLGYEGYKNVMENCQENASVLREGLEKTGRFNIISKEIGVPLVAFSLKDNRQHNEFEISETLRRFGWIVPAYTMPPNAQHITVLRVVIREDFSRTLAERLVRDIEKVLHELDTLPARVNAKLAVAEEQAAANGSEVHKKTDSEVQLEMITAWKKFVEEKKKKTNRVC

SEQ ID NO: 2 - protein, 496 aa, Nicotiana tabacum
MVLSKTASESDVSIHSTFASRYVRTSLPRFKMPENSIPKEAAYQIINDELMLDGNPRLNLASFVTTWMEPECNTLMMDSINKNYVDMDEYPVTTELQNRCVNMIAHLFNAPLGDGETAVGVGTVGSSEAIMLAGLAFKRKWQNKMKAQGKPFDKPNIVTGANVQVCWEKFARYFEVELKEVKLSDGYYVMDPEKAVEMVDENTICVAAILGSTLNGEFEDVKRLNDLLIEKNKETGWDTPIHVDAASGGFIAPFLYPELEWDFRLPLVKSINVSGHKYGLVYAGIGWAIWRNKEDLPDELIFHINYLGADQPTFTLNFSKGSSQVIAQYYQLIRLGFEGYKNVMENCQENARVLREGIEKSGRFNIISKEIGVPLVAFSLKDNSQHNEFEISETLRRFGWIVPAYTMPPNAQHVTVLRVVIREDFSRTLAERLVIDIEKVLHELDTLPARVNAKLAVAEANGSGVHKKTDREVQLEITTAWKKFVADKKKKTNGVC

SEQ ID NO: 3 - protein, 499 aa, Beta vulgaris
MVLSSAVTESDNSVHSTFASRYVRVCLPRFQMPDKSIPKEAAYQIINDELMLDGNPRLNLASFVTTWMEPECDKLMMDAINKNYVDMDEYPVTTELQNRCVNMIANLFNAPLGDSEVAVGVGTVGSSEAIMLAGLAFKRRWQNKRKALGLPTDKPNIVTGANVQVCWEKFARYFEVELKEVKLSDGYYVMDPEKAVEMVDENTICVAAILGSTLNGEFEDVKLLNDLLVAKNKETGWDTPIHVDAASGGFIAPFIYPELEWDFRLPLVKSINVSGHKYGLVYAGVGWVIWRSKEDLPEELIFHINYLGADQPTFTLNFSKGSSQIIAQYYQLIRLGFEGYKNIMENCQENAMILKQGIEKTDRFNIISKDMGVPLVAFSLKDNSLHNEFEISEHLRKFGWIVPAYTMPPDAQHVTVLRVVIREDFSRTLADRLVNDLQKVLNELDALPARANAKLNVPHAVDGENQASRKKTALEMQMEVCNVWKKFVDDRKLKTNGVC

SEQ ID NO: 4 - protein, 502 aa, Beta vulgaris
MVLSETSADASTQNLSCTFASRYVRTALPRFKMPEKSIPKDAAYQIINDELMLDGNPRLNLASFVTTWMEPECDQLIMASINKNYVDMDEYPVTTELQNRCVNIIAHLFNAPIGEEETAVGVGTVGSSEAIMLAGLAFKRKWQQKRRAEGKPCDKPNIVTGANVQVCWEKFARYFEVELKEVKLKEGYYVMDPEKAVDMVDENTICVAAILGSTLTGEFEDVKLLNELLTKKNKETGWDTPIHVDAASGGFIAPFIYPDIEWDFRLPLVKSINVSGHKYGLVYAGIGWVVWRSKDDLPEDLIFHINYLGTDQPTFTLNFSKGSSQIIAQYYQLIRLGFEGYKNVMENCRDTAKMLKEEIEKTGRFNIVSKDVGVPVVAFSLKDSSSNYTVFDIAENLRRFGWIVPAYTMPADAEHVAVLRVVVREDFSRSLADRLVSNIQKVLDEMHADPVPVSSKSAHVTLDKDETQGNKAVIKKDVVEIQKDITKYWKKLVDRSKARVVC

SEQ ID NO: 5 - protein, 493 aa, Glycine max
MVFPKPSSESDLFLCSNFASRYARDSLPRFSMPENSMPKEAAYQNIHDELQLDAIPKLNLASFVTTSMEEECNKLIMESINKNYVDMDEYPATTDLHNRCVNMIARMFHAEIGENENATGAGTVGSSEAIMLAGLAFKKKWQNKRKAEGKPYDKPNLVTGSNVQVCWEKFARYFEVELREVEVREGYYVMDPAKAVELVDENTICVAAILGSTYNGEFEDVKLLNDLLLEKNKQTGWDTPIHVDAASGGFIAPFLYPELEWDFRLPLVKSINVSGHKYGLVYAGIGWVIWRTKDDLPEDLVFHINYLGADQPTFTLNFSKGSSQIIAQYYQLIRLGHEGYRSIMENCRDNAMVLKESLEKSGHFNILSKDNGVPVVAFSLKDRSQYDEYKISEMLRRHGWIVPAYPMPPAAQHINVLRVVIRAEFSRTLAERLVFDIYNVLHELEKLHPPKVTNFIKEENKALVETGVKKTALDAHREIIAQESNKRQKIMAA

SEQ ID NO: 6 - protein, 503 aa, Glycine max
MVLSKTTSESDVSVHSTFASRYVRTSLPRFKMPEESIPKEAAYQIINDELMLDGNPRLNLASFVTTWMEPECDKLIMAAINKNYVDMDEYPVTTELQNRCVNMIAHLFNAPLEETEAAVGVGTVGSSEAIMLAGLAFKRKWQNRRKQEGKPFDKPNIVTGANVQVCWEKFARYFEVELKEVKLRDDYYVMDPEKAVELVDENTICVAAILGSTLNGEFEDVKRLNDLLIEKNKITGWDTPIHVDAASGGFIAPFLYPELEWDFRLPLVKSINVSGHKYGLVYAGIGWVIWRSKEDLPEELIFHINYLGADQPTFTLNFSKGSSQVIAQYYQLIRLGFEGYRNVMENCRDNMLVLKEGLEKTGRFSIVSKDNGVPLVAFTLKDHTHFDEFQISDFLRRFGWIVPAYTMPPDAQHVTVLRVVIREDFSRTLAERLVADVEKVLHELDSLPARVISSTSVTVTAEENGKVVVAKKSAMETQREITAIWKKFVLERKKNNDKMNGVC

SEQ ID NO: 7 - protein, 492 aa, Oryza sativa
MVLSHGVSGSDESVHSTFASRYVRTSLPRFRMPEQSIPKEAAYQIINDELMLDGNPRLNLASFVTTWMEPECDKLIQASVNKNYVDMDEYPVTTELQNRCVNMIAHLFNAPLGDSETAVGVGTVGSSEAIMLAGLAFKRRWQNKMKAAGKPCDKPNIVTGANVQVCWEKFARYFEVELKEVKLSDGYYVMDPAKAVDMVDENTICVAAILGSTLNGEFEDVKLLNDLLTKKNAETGWDTPIHVDAASGGFIAPFLYPELEWDFRLPLVKSINVSGHKYGLVYAGIGWCIWRSKEDLPEELIFHINYLGADQPTFTLNFSKGSSQVIAQYYQLIRLGFEGYKNIMENCQENAMVLKQGLEKTGRFNIVSKDNGVPLVAFSLKDSARHNEFEISDFLRRFGWIVPAYTMPPDAQHVTVLRVVIREDFSRTLAERLVLDVEKVLHELDALPARVVANGGDAAAASASEREMEKQREVISLWKRAVLAKKKTNGVC

SEQ ID NO: 8 - protein, 500 aa, Oryza sativa
MVLTHVEAVEEGSEAAAAVFASRYVQDPVPRYELGERSISKDAAYQIVHDELLLDSSPRLNLASFVTTWMEPECDRLILEAINKNYADMDEYPVTTELQNRCVNIIARLFNAPVGDGEKAVGVGTVGSSEAIMLAGLAFKRRWQNRRKAAGKPHDKPNIVTGANVQVCWEKFARYFEVELKEVKLTEGCYVMDPVKAVDMVDENTICVAAILGSTLTGEFEDVRRLNDLLAAKNKRTGWDTPIHVDAASGGFIAPFIYPELEWDFRLPLVKSINVSGHKYGLVYAGVGWVIWRNKEDLPEELIFHINYLGADQPTFTLNFSKGSSQIIAQYYQFLRLGFEGYKSVMKNCMESARTLREGLEKTGRFTIISKEEGVPLVAFTFKDGAGAQAFRLSSGLRRYGWIVPAYTMPAALEHMTVLRVVVREDFGRPLAERFLSHVRMALDEMDLAARAPVPRVQLTIELGPARTAGEEASIRVVKSEAVPVRKSVPLVAGKTKGVC

SEQ ID NO: 9 - protein, 493 aa, Zea mays
MVLSHGVGGSDESVHSTFASRYVRTSLPRYRMPEQSIPKEAAYQIINDELMLDGNPRLNLASFVTTWMEPECDKLIQASVNKNYVDMDEYPVTTELQNRCVNMIAHLFNAPLGDAETAVGVGTVGSSEAIMLAGLAFKRRWQNKMKAAGKPCDKPNIVTGANVQVCWEKFARYFEVELKEVKLSDGYYVMDPQKAVDMVDENTICVAAILGSTLNGEFEDVKLLNDLLTKKNAETGWDTPIHVDAASGGFIAPFLYPELEWDFRLPLVKSINVSGHKYGLVYAGIGWCIWRTKVDLPEELIFHINYLGADQPTFTLNFSKGSSQVIAQYYQLIRLGFEGYKNIMENCQENATVLKQGLEKTGKFNIVSKDNGVPLVAFSLKDSSRHSEFEISDFLRRFGWIVPAYTMPPDAQHVTVLRVVIREDFSRTLAERLVLDIEKVLHELDALPARVPSGDLAALAAAESSEREMEKQRQVISLWKRAVLAKKKTNGVC

SEQ ID NO: 10 - protein, 490 aa, Zea mays
MVLSKAVSESDMSVHSTFASRYVRSSLPRYRMPENSIPKEAAYQIINDELMLDGNPRLNLASFVTTWMEPECDKLIMAAINKNYVDMDEYPVTTELQNRCVNMIAHLFNAPLGESETAVGVGTVGSSEAIMLAGLAFKRRWQNKRRAEGKPFDKPNIVTGANVQVCWEKFARYFEVELREVKLRDGYYVMDPEKAVEMVDENTICVAAILGSTLNGEFEDVKLLNDLLQAKNRETRWETPIHVDAASGGFIAPFLYPDLEWDFRLPLVKSINVSGHKYGLVYAGIGWCIWRSKEDLPEELIFHINYLGADQPTFTLNFSKGSSQVIAQYYQLIRHGFEGYRNIMENCHENAMVLKEGLEKTGRFDIVSKDEGVPLVAFSLKDRSRHDEFEISDMLRRFGWIVPAYTMPPDAQHVTVLRVVIREEFSRTLAERLVLDIEKVMYQLDALPSRLPPPPPAAPLLRKKTELETQRSVTEAWKKFVLAKKTNGVC

SEQ ID NO: 11 - protein, 466 aa, Escherichia coli
MDQKLLTDFRSELLDSRFGAKAISTIAESKRFPLHEMRDDVAFQIINDELYLDGNARQNLATFCQTWDDENVHKLMDLSINKNWIDKEEYPQSAAIDLRCVNMVADLWHAPAPKNGQAVGTNTIGSSEACMLGGMAMKWRWRKRMEAAGKPTDKPNLVCGPVQICWHKFARYWDVELREIPMRPGQLFMDPKRMIEACDENTIGVVPTFGVTYTGNYEFPQPLHDALDKFQADTGIDIDMHIDAASGGFLAPFVAPDIVWDFRLPRVKSISASGHKFGLAPLGCGWVIWRDEEALPQELVFNVDYLGGQIGTFAINFSRPAGQVIAQYYEFLRLGREGYTKVQNASYQVAAYLADEIAKLGPYEFICTGRPDEGIPAVCFKLKDGEDPGYTLYDLSERLRLRGWQVPAFTLGGEATDIVVMRIMCRRGFEMDFAELLLEDYKASLKYLSDHPKLQGIAQQNSFKHT

SEQ ID NO: 12 - protein, 466 aa, Escherichia coli
MDKKQVTDLRSELLDSRFGAKSISTIAESKRFPLHEMRDDVAFQIINDELYLDGNARQNLATFCQTWDDENVHKLMDLSINKNWIDKEEYPQSAAIDLRCVNMVADLWHAPAPKNGQAVGTNTIGSSEACMLGGMAMKWRWRKRMEAAGKPTDKPNLVCGPVQICWHKFARYWDVELREIPMRPGQLFMDPKRMIEACDENTIGVVPTFGVTYTGNYEFPQPLHDALDKFQADTGIDIDMHIDAASGGFLAPFVAPDIVWDFRLPRVKSISASGHKFGLAPLGCGWVIWRDEEALPQELVFNVDYLGGQIGTFAINFSRPAGQVIAQYYEFLRLGREGYTKVQNASYQVAAYLADEIAKLGPYEFICTGRPDEGIPAVCFKLKDGEDPGYTLYDLSERLRLRGWQVPAFTLGGEATDIVVMRIMCRRGFEMDFAELLLEDYKASLKYLSDHPKLQGIAQQNSFKHT

SEQ ID NO: 13 - protein, 473 aa, Artificial Sequence
  Truncated GAD Petunia
MVLSKTVSQSDVSIHSTFASRYVRTSLPRFKMPDNSIPKEAAYQIINDELMLDGNPRLNLASFVTTWMEPECDKLMMDSINKNYVDMDEYPVTTELQNRCVNMIAHLFNAPLEDGETAVGVGTVGSSEAIMLAGLAFKRKWQNKMKAQGKPCDKPNIVTGANVQVCWEKFARYFEVELKEVKLSEGYYVMDPEKAVEMVDENTICVAAILGSTLNGEFEDVKRLNDLLVEKNKETGWDTPIHVDAASGGFIAPFIYPELEWDFRLPLVKSINVSGHKYGLVYAGIGWVVWRNKDDLPDELIFHINYLGADQPTFTLNFSKGSSQVIAQYYQLIRLGYEGYKNVMENCQENASVLREGLEKTGRFNIISKEIGVPLVAFSLKDNRQHNEFEISETLRRFGWIVPAYTMPPNAQHITVLRVVIREDFSRTLAERLVRDIEKVLHELDTLPARVNAKLAVAEEQAAANGSEVHKKT

SEQ ID NO: 14 - protein, 459 aa, Artificial Sequence
  Truncated GAD Tobacco
MVLSKTASESDVSIHSTFASRYVRTSLPRFKMPENSIPKEAAYQIINDELMLDGNPRLNLASFVTTWMEPECNTLMMDSINKNYVDMDEYPVTTELQNRCVNMIAHLFNAPLGDGETAVGVGTVGSSEAIMLAGLAFKRKWQNKMKAQGKPFDKPNIVTGANVQVCWEKFARYFEVELKEVKLSDGYYVMDPEKAVEMVDENTICVAAILGSTLNGEFEDVKRLNDLLIEKNKETGWDTPIHVDAASGGFIAPFLYPELEWDFRLPLVKSINVSGHKYGLVYAGIGWAIWRNKEDLPDELIFHINYLGADQPTFTLNFSKGSSQVIAQYYQLIRLGFEGYKNVMENCQENARVLREGIEKSGRFNIISKEIGVPLVAFSLKDNSQHNEFEISETLRRFGWIVPAYTMPPNAQHVTVLRVVIREDFSRTLAERLVIDIEKVLHELDTLPARVNAKLAVAE

SEQ ID NO: 15 - DNA, 25 bp, Artificial Sequence
  synthetic single-stranded oligonucleotide or guiding RNA core sequence
gcttctaggaagtagactgccttgg

SEQ ID NO: 16 - DNA, 29 bp, Artificial Sequence
  oligonucleotide primer
ggatggtgaaaatcaagcttctaggaagt

SEQ ID NO: 17 - DNA, 31 bp, Artificial Sequence
  oligonucleotide primer
ccatgatcatactcaactaaacctcactccc

SEQ ID NO: 18 - DNA, 30 bp, Artificial Sequence
  oligonucleotide primer
tttggttggattgtaccagcttacaccatg

SEQ ID NO: 19 - DNA, 30 bp, Artificial Sequence
  oligonucleotide primer
gacctccatttgcatctccaaggcagtcta

SEQ ID NO: 20 - DNA, 25 bp, Artificial Sequence
  synthetic single-stranded oligonucleotide or guiding RNA core sequence
atggaggtctgctaatgtttggaag

SEQ ID NO: 21 - DNA, 28 bp, Artificial Sequence
  synthetic single-stranded oligonucleotide or guiding RNA core sequence
aatgttccacactaggctgtggatggtg

SEQ ID NO: 22 - DNA, 25 bp, Artificial Sequence
  synthetic single-stranded oligonucleotide or guiding RNA core sequence
gctgttatcaaataagatgtggtag

SEQ ID NO: 23 - DNA, 25 bp, Artificial Sequence
  synthetic single-stranded oligonucleotide or guiding RNA core sequence
aaggacataacctaagtactggaaa

SEQ ID NO: 24 - DNA, 25 bp, Artificial Sequence
  synthetic single-stranded oligonucleotide or guiding RNA core sequence
gtcacccttgactaaggatgaaaca

SEQ ID NO: 25 - DNA, 25 bp, Artificial Sequence
  synthetic single-stranded oligonucleotide or guiding RNA core sequence
gtagtggttgcttagaagagtgcta

SEQ ID NO: 26 - DNA, 25 bp, Artificial Sequence
  synthetic single-stranded oligonucleotide or guiding RNA core sequence
gagactcagaggtaaatcactgcca

SEQ ID NO: 27 - DNA, 25 bp, Artificial Sequence
  synthetic single-stranded oligonucleotide or guiding RNA core sequence
acagtcactgcttaagaaaatggca

SEQ ID NO: 28 - DNA, 25 bp, Artificial Sequence
  synthetic single-stranded oligonucleotide or guiding RNA core sequence
agggagatggagtagcagcgcgagg

SEQ ID NO: 29 - DNA, 25 bp, Artificial Sequence
  synthetic single-stranded oligonucleotide or guiding RNA core sequence
aagcagcgcgagtgatctccctctg

SEQ ID NO: 30 - DNA, 27 bp, Artificial Sequence
  synthetic single-stranded oligonucleotide or guiding RNA core sequence
ggcggcgacgcctaggccgcgtcggcg

SEQ ID NO: 31 - DNA, 25 bp, Artificial Sequence
  synthetic single-stranded oligonucleotide or guiding RNA core sequence
ctgctcaggaaatagacggagctgg

SEQ ID NO: 32 - DNA, 26 bp, Artificial Sequence
  synthetic single-stranded oligonucleotide or guiding RNA core sequence
ctggagacgcagtaggtcggtcacgg

SEQ ID NO: 33 - DNA, 27 bp, Artificial Sequence
  synthetic single-stranded oligonucleotide or guiding RNA core sequence
cctcccccggcgtagccgctgctcagg

SEQ ID NO: 34 - DNA, 25 bp, Artificial Sequence
  synthetic single-stranded oligonucleotide or guiding RNA core sequence
gccgagtccagctagagggagatgg

SEQ ID NO: 35 - DNA, 25 bp, Artificial Sequence
  synthetic single-stranded oligonucleotide or guiding RNA core sequence
aagcagcgccagtgatctcgctctg

SEQ ID NO: 36 - DNA, 29 bp, Artificial Sequence
  synthetic single-stranded oligonucleotide or guiding RNA core sequence
gacctagccgcgtaggccgcggccgagtc

SEQ ID NO: 37 - DNA, 25 bp, Beta vulgaris
gcttctaggaagaagactgccttgg

SEQ ID NO: 38 - DNA, 24 bp, Beta vulgaris
atggaggtctgcaatgtttggaag

SEQ ID NO: 39 - DNA, 28 bp, Beta vulgaris
aatgttccacacgctgtggatggtgaaa

SEQ ID NO: 40 - DNA, 25 bp, Beta vulgaris
gctgttatcaaaaaagatgtggtag

SEQ ID NO: 41 - DNA, 24 bp, Beta vulgaris
aaggacataaccaagtactggaaa

SEQ ID NO: 42 - DNA, 24 bp, Beta vulgaris
gtcacccttgacaaggatgaaaca

SEQ ID NO: 43 - DNA, 25 bp, Glycine max
gtagtggttgctaagaagagtgcta

SEQ ID NO: 44 - DNA, 25 bp, Glycine max
gagactcagagggaaatcactgcca

SEQ ID NO: 45 - DNA, 25 bp, Glycine max
acagtcactgctgaagaaaatggca

SEQ ID NO: 46 - DNA, 25 bp, Oryza sativa
agggagatggagaagcagcgcgagg

SEQ ID NO: 47 - DNA, 26 bp, Oryza sativa
aagcagcgcgaggtgatctccctctg

SEQ ID NO: 48 - DNA, 27 bp, Oryza sativa
ggcggcgacgccgccgccgcgtcggcg

SEQ ID NO: 49 - DNA, 25 bp, Zea mays
ctgctcaggaaaaagacggagctgg

SEQ ID NO: 50 - DNA, 25 bp, Zea mays
ctggagacgcagaggtcggtcacgg

SEQ ID NO: 51 - DNA, 27 bp, Zea mays
cctcccccggcggcgccgctgctcagg

SEQ ID NO: 52 - DNA, 25 bp, Zea mays
gccgagtccagcgagagggagatgg

SEQ ID NO: 53 - DNA, 26 bp, Zea mays
aagcagcgccaggtgatctcgctctg

SEQ ID NO: 54 - DNA, 29 bp, Zea mays
gacctagccgcgctcgccgcggccgagtc